(19) United States
(12) Patent Application Publication
(10) Pub. No.: US 2024/0353431 A1
(43) Pub. Date: Oct. 24, 2024

(54) SELECTIVE MEASUREMENT OF HUMAN FACTOR VIII

(71) Applicant: TAKEDA PHARMACEUTICAL COMPANY LIMITED, Osaka (JP)

(72) Inventors: Alfred Weber, Vienna (AT); Herbert GRITSCH, Vienna (AT); Gerald SCHRENK, Vienna (AT); Manfred BILLWEIN, Vienna (AT); Andrea ENGELMAIER, Vienna (AT); Christopher ZLABINGER, Vienna (AT); Michael DOCKAL, Vienna (AT)

(73) Assignee: TAKEDA PHARMACEUTICAL COMPANY LIMITED, Osaka-Shi, Osaka (JP)

(21) Appl. No.: 18/684,541

(22) PCT Filed: Aug. 20, 2021
(86) PCT No.: PCT/IB2021/000578

§ 371 Date: Feb. 16, 2024

(51) Int. Cl. G01N 33/86

(52) U.S. Cl. CPC G01N 33/86; G01N 2333/755

(57) ABSTRACT

Described herein are methods and kits for selective and sensitive activity measurement of human coagulation factor VIII (FVIII).

### FIELD OF THE INVENTION

This invention relates generally to the field of chromogenic-based assays and more specifically to chromogenic-based assays for the measurement of factor VIII (FVIII) procoagulant activity in plasma and other fluids.

### BACKGROUND

The ability to selectively measure the procoagulant activity of therapeutically administered factor VIII (FVIII) in plasma or other fluids is essential for examining a factor VIII replacement treatment approach. During the testing and development of human FVIII drug candidates, or the evaluation of alternative administration routes, it is often required to test them in animal models having normal levels of endogenous FVIII. It is challenging, however, to quantify human FVIII and differentiate it with sufficient accuracy and precision from the endogenous FVIII of the animal host model. Not only can the presence of endogenous non-human FVIII potentially biases the pharmacokinetic study analysis, certain manufacturing process-related additives can also impact the assay performance. For example, solvent/detergent mixtures used during the viral inactivation process may interfere with the assay.

Accordingly, there is an unmet need for assays that allow selective and sensitive activity measurement of human FVIII in the presence of animal plasma containing non-human FVIII or in the presence of manufacturing-related additives.

### SUMMARY OF THE INVENTION

Described herein are methods and kits that allow selective and sensitive activity measurement of human coagulation factor VIII (FVIII). The methods utilize an FVIII recognizing capture agent that selectively binds to human FVIII, even in the presence of endogenous FVIII from another animal and/or other manufacturing-related additives.

In one aspect, provided herein is a method for measuring the activity of human coagulation factor VIII (FVIII) in a sample, comprising the steps of:
a) incubating the sample with a capture agent that selectively binds the human FVIII under conditions and for a time sufficient to allow the capture agent to selectively bind to the human FVIII, whereby forming a reaction mixture, wherein the capture agent is attached to a solid support:
b) removing unbound fraction of human FVIII from the reaction mixture; and
c) measuring the activity of human FVIII in the reaction mixture using a chromogenic assay.

In some embodiments, the method further comprises d) determining the activity of human FVIII in the reaction mixture by comparing with an FVIII reference preparation.

In some embodiments, the chromogenic assay is performed by
c1) adding to the reaction mixture activated factor IX (FIXa), factor X (FX), calcium ion, phospholipids and thrombin and incubating the reaction mixture under conditions and for a time sufficient to generate activated FX (FXa):
c2) adding to the reaction mixture an FXa specific chromogenic substrate and incubating the reaction mixture under conditions and for a time sufficient to generate a measurable signal:
c3) optionally, adding to the reaction mixture a stopping agent to stop signal generation from the FXa specific chromogenic substrate; and
c4) measuring the signal generated from the reaction mixture.

In some embodiments, the chromogenic substrate is CH₃OCO-D-cyclohexylalanine-Gly-Arg-pNA.

In some embodiments, the stopping agent is acetic acid.

In some embodiments, the capture agent selectively binds to the A1, A2, A3, B, C1, or C2 domain of human FVIII. In some embodiments, the capture agent selectively binds to the A2 domain of human FVIII. In some embodiments, the capture agent does not bind to the B domain of human FVIII. In some embodiments, the capture agent is an antibody, an antigen-binding fragment, an affibody, an aptamer or an affinity ligand.

In some embodiments, the capture antibody is GMA-8024, GMA-8023, or an antigen-binding fragment or a variant thereof.

In some embodiments, the sample comprises plasma and/or serum from a non-human animal. In some embodiments, the non-human animal is a laboratory animal. In some embodiments, the non-human animal is sheep, goat, cynomolgus monkey, rabbit, rat, rhesus macaque, mouse, swine, hamster, minipig, or guinea pig. In some embodiments, the sample comprises citrated plasma.

In some embodiments, the sample is diluted prior to step a). The sample may be is diluted by a factor of between 1:2 to about 1:20000. In one embodiment, the sample is diluted by a factor of about 1:10.

In some embodiments, the solid support is a flask, ELISA plate, multi-well plate, a film, a tube, a sheet, a column, or a microparticle.

In some embodiments, the incubation in step a) is performed for about 1 hour at room temperature.

In some embodiments, the incubation in step c1) is performed for about 15 min at room temperature.

In some embodiments, the incubation in step c2) is performed for about 25 min at room temperature.

In some embodiments, the removal of unbound human FVIII in step b) is performed by washing the solid support with a washing buffer. In some embodiments, the removal of unbound human FVIII in step b) is performed by washing the solid support with PBS-Tween buffer for about three times.

In various embodiments, the human FVIII is a recombinant FVIII. In some embodiments, the human FVIII is full-length FVIII. In some embodiments, the human FVIII is B-domain deleted FVIII.

In one aspect, provided herein is a kit for measuring the activity of human coagulation factor VIII (FVIII) in a sample, comprising
i. a capture agent that selectively binds the human FVIII, wherein the capture agent is attached to a solid support:
ii. a first vial comprising one or more of activated factor IX (FIXa), factor X (FX), calcium ion, phospholipids and thrombin;
iii. a second vial comprising an FXa specific chromogenic substrate; and/or
iv. optionally, a third vial comprising a stopping agent.

In some embodiments of the kit described above, the capture agent selectively binds to the A1, A2, A3, B, C1, or C2 domain of human FVIII. In some embodiments of the kit described above, the capture agent selectively binds to the A2 domain of human FVIII. In some embodiments of the kit described above, the capture agent does not bind to the B domain of human FVIII. In some embodiments, the capture agent is an antibody, an antigen-binding fragment, an affibody, an aptamer or an affinity ligand. In some embodiments, the capture antibody is GMA-8024, GMA-8023, or an antigen-binding fragment or a variant thereof.

In some embodiments of the kit described above, the solid support is a flask, ELISA plate, multi-well plate, a film, a tube, a sheet, a column, or a microparticle. In some embodiments, the chromogenic substrate is CH₃OCO-D-cyclohexylalanine-Gly-Arg-pNA. In some embodiments, the stopping agent is acetic acid.

Some embodiments of the kit may further comprise one or more of a reaction buffer, an FVIII dilution buffer, a washing buffer and/or an FVIII reference preparation.

### BRIEF DESCRIPTION OF THE DRAWINGS

The accompanying Figures, which are incorporated in and constitute a part of this specification, illustrate several aspects described below.

FIG. 1 shows a schematic of a chromogenic FVIII activity assay according to the invention. CHA and pNA stand for cyclohexylalanine and p-nitrophenylanilide, respectively.

FIG. 2 shows the blank-corrected optical densities (ODs) obtained for the B-domain deleted (BDD) rFVIII concentrations of 188, 94 and 47 mU/mL. The FVIII activity measured in mIU/mL of the different capturing antibodies tested according to an embodiment of the invented chromogenic FVIII activity assay. From left to right, the anti-FVIII capturing antibodies are: GMA-8024, GMA-8023, GMA-012, ESH-8, ESH-4, RFF-VIIIC/8, RFF-VIIIC/5, and N55195M.

FIG. 3 shows the mean calibration curve and the agreement of the back-fitted assay calibrations to their nominal values. Error bars mark the single standard deviation of the means.

FIG. 4 shows the mean calibration curve obtained by using the BDD IFVIII preparation and the agreement of the back-fitted assay calibrations to their nominal values. Error bars mark the single standard deviation of the means.

FIG. 5 shows the individual recoveries determined for the human FVIII (100) mU/mL) spiked to the citrated animal plasma species. Numbers on top of the columns indicate the recovery as a percent of the spiked 100 mU/mL.

FIG. 6 shows the recovery determined for the human FVIII (100 mU/mL) spiked to the animal serum samples. Numbers on top of the columns indicate the recovery as a percent of the spiked 100 mU/mL.

FIG. 7 shows the dose-response curves for human FVIII spiked to citrated goat, sheep and cynomolgus monkey plasma. The insert shows the relative slopes of the double logarithmic concentration-response curves, given as a percent of that determined for the buffer dilution series, and the curves' corresponding coefficients of determination.

FIG. 8 shows the dose-response curves for human FVIII spiked to citrated rabbit and rat plasma and mouse serum. The insert shows the relative slopes of the double logarithmic concentration-response curves, given as a percent of that determined for the buffer dilution series, and the curves' corresponding coefficients of determination.

FIG. 9) shows the dose-response curves for human FVIII spiked to citrated rhesus monkey plasma and guinea pig serum. The insert shows the relative slopes of the double logarithmic concentration-response curves, given as a percent of that determined for the buffer dilution series, and the curves' corresponding coefficients of determination.

FIG. 10 shows the dose-response curves for human FVIII spiked to swine and hamster serum. The insert shows the relative slopes of the double logarithmic concentration-response curves, given as a percent of that determined for the buffer dilution series, and the curves' corresponding coefficients of determination.

FIGS. 11A-11D show the data of 108 measurements, carried out by three operators over a period of six months, to demonstrate the intermediate precision and robustness of the assay over time. FIGS. 11A and 11B show all individual values and those of the three operators, respectively (mean±SD), while FIGS. 11C and 11D present the data in form of a QQ plot (11C) and of a histogram (11D).

FIG. 12 shows the concentration-response curves obtained for a sample containing 10 IU human FVIII/mL in the presence of the solvent detergent (S/D) solution, i.e., 1% Triton X 100, 0.3% Polysorbate 80 and 0.3% solvent detergent (S/D), and the same FVIII concentration in PBS buffer (=native FVIII). The insert shows the relative slopes of the dilution-response curves, expressed as a percent of the slope determined for native FVIII, and their coefficients of variation.

FIG. 13 shows the concentration-response curves obtained using a FVIII concentration of 1 IU/mL in the presence of the solvent detergent (S/D) solution, i.e., 1% Triton X 100, 0.3% Polysorbate 80 and 0.3% solvent detergent (S/D) and the same FVIII concentration in PBS buffer (=native FVIII), requiring a 10-times lower sample dilution and thus markedly increasing the levels of S/D components during the antibody-based FVIII capture. The insert shows the relative slopes of the dilution-response curves, expressed as a percent of the slope determined for native FVIII, and their coefficients of variation.

FIG. 14 shows the recovery of three levels of human FVIII (10, 1 and 0.1 IU/mL) in S/D solution and the individual components of the S/D solution.

FIG. 15 shows assay performance in presence of individual components of the S/D virus inactivation solution.

### DETAILED DESCRIPTION

The present application provides, among other things, methods and kits for the measuring the procoagulant activity of human coagulation factor VIII (FVIII) in a sample. As demonstrated in the Examples section below, the inventors investigated whether the procoagulant activity of human factor VIII (FVIII) can be selectively measured in an animal plasma matrix containing endogenous FVIII by combining selective capture of human FVIII followed by a chromogenic assay for FVIII activity, carried out not in solution (as with conventional assays) but with the captured and, thus, purified human FVIII. The methods can also be used for ex-vivo plasma testing where FVIII products are administered in addition to the endogenous FVIII present in the animal. Furthermore, the assay can be used for manufacturing-related matrices, in which FVIII measurement may not be feasible due to assay interference. In a clinical setting, finally, the assay can enable the measurement of human FVIII in the presence of FVIII activity-mimicking agents, like bispecific antibodies, without interference of these agents.

In one aspect, provided herein is a method for measuring the activity of human coagulation FVIII in a sample. Steps of the method can include one or more of: a) incubating the sample with a capture agent that selectively binds human FVIII under conditions and for a time sufficient to allow the capture agent to selectively bind to the human FVIII, whereby forming an reaction mixture, wherein the capture agent is attached to a solid support; b) removing unbound human FVIII from the reaction mixture; and c) measuring the activity of human FVIII in the reaction mixture using an activity assay.

In some embodiments, the method can further include step d) determining the activity of human FVIII in the reaction mixture by comparing with an FVIII reference preparation.

Aspects of the present specification disclose a capture agent. A capture agent refers to any molecule capable of selective or substantially selective (that is with limited cross-reactivity) binding to human FVIII, or a target site present on human FVIII or otherwise associating with human FVIII. As used herein, the term “selectively” refers to having a unique effect or influence or reacting in only one way or with only one target. As used herein, the term “selectively binds,” when made in reference to a capture agent, refers to the discriminatory binding of the capture agent to human FVIII or the indicated target site present on human FVIII such that the agent does not substantially cross react with non-target sites on human FVIII or FVIII from other animals. Any capture agent that can selectively bind to human FVIII, or a target site present on human FVIII or otherwise associating with human FVIII may be used in the methods disclosed herein. A capture agent generally has a single specificity although capture agents having multiple specificities for two or more target sites may be used. Non-limiting examples of a capture agent include an antibody, an antigen-binding fragment, an affibody, an aptamer (e.g., DNA aptamer), a synthetic peptide, a binding molecule, a nucleic acid, and other affinity ligands capable of specifically capturing human FVIII, and preferably not interfering with the activity assay, e.g., interfering with FVIII's role as cofactor for FIXa-mediated FXa generation.

In some embodiments, the capture agent selectively binds to the A1, A2, A3, B, C1. or C2 domain of human FVIII. In some embodiments, the capture agent selectively binds to the A2 domain of human FVIII. In some embodiments, the capture agent does not bind to the B domain of human FVIII.

Selective binding of a capture agent can include binding properties such as, e.g., binding affinity, binding specificity, and binding avidity. Binding affinity refers to the length of time a capture agent resides at its binding site or moiety, and can be viewed as the strength with which a capture agent binds its binding site or moiety. Binding affinity can be described a capture agent's equilibrium dissociation constant (KD), which is defined as the ratio Kd/Ka at equilibrium, where Ka is a capture agent's association rate constant and Kd is a capture agent's dissociation rate constant. Binding affinity is determined by both the association and the dissociation and alone neither high association nor low dissociation can ensure high affinity. The association rate constant (Ka), or on-rate constant (Kon), measures the number of binding events per unit time, or the propensity of a capture agent's and its binding site or moiety to associate reversibly into its agent-moiety complex. The association rate constant is expressed in M⁻¹s⁻¹, and is symbolized as follows: [CA]×[BS]×Kon. The larger the association rate constant, the more rapidly a capture agent binds to its binding site or moiety, or the higher the binding affinity between a capture agent and its binding site or moiety. The dissociation rate constant (Kd), or off-rate constant (Koff), measures the number of dissociation events per unit time propensity of agent-moiety complex to separate (dissociate) reversibly into its component molecules, namely the capture agent and its binding site or moiety. The dissociation rate constant is expressed in s⁻¹, and is symbolized as follows: [CA+BS]×Koff. The smaller the dissociation rate constant, the more tightly bound a capture agent is to its binding site or moiety, or the higher the binding affinity between capture agent and its binding site or moiety. The equilibrium dissociation constant (KD) measures the rate at which new agent-moiety complexes formed equals the rate at which agent-moiety complexes dissociate at equilibrium. The equilibrium dissociation constant is expressed in M, and is defined as Koff/Kon=[CA]>[BS]/[CA+BS], where [CA] is the molar concentration of a capture agent, [BS] is the molar concentration of the binding site or moiety, and [CA+BS] is the of molar concentration of the agent-moiety complex, where all concentrations are of such components when the system is at equilibrium. The smaller the equilibrium dissociation constant, the more tightly bound a capture agent is to its binding site or moiety, or the higher the binding affinity between a capture agent and its binding site or moiety.

In an embodiment, the binding affinity of a capture agent disclosed herein may have an association rate constant of, e.g., less than 1×10⁵M⁻¹s⁻¹, less than 1×10⁶M⁻¹s⁻¹, less than 1×10⁷M⁻¹s⁻¹, or less than 1×10⁸. In another embodiment, the binding affinity of a capture agent disclosed herein may have an association rate constant of, e.g., more than 1×10⁵M⁻¹s⁻¹, more than 1×10⁶M⁻¹s⁻¹, more than 1×10⁷M⁻¹s⁻¹, or more than 1×10⁸M⁻¹s⁻¹. In other aspects, the binding affinity of a capture agent disclosed herein may have an association rate constant between 1×10⁵M⁻¹s⁻¹ to 1×10⁸M⁻¹s⁻¹, 1×10⁶M⁻¹s⁻¹ to 1×10⁸M⁻¹s⁻¹, 1×10⁵M⁻¹s⁻¹ to 1×10⁷M⁻¹s⁻¹, or 1×10⁶M⁻¹s⁻¹ to 1×10⁷M⁻¹s⁻¹.

In another embodiment, the binding affinity of a capture agent disclosed herein may have a disassociation rate constant of less than 1×10⁻³ s⁻¹, less than 1×10⁻⁴ s⁻¹, or less than 1×10⁻⁵ s⁻¹. In other aspects of this embodiment, the binding affinity of a capture agent disclosed herein may have a disassociation rate constant of, e.g., less than 1.0×10⁻⁴ s⁻¹, less than 2.0×10⁻⁴ s⁻¹, less than 3.0×10⁴ s⁻¹, less than 4.0×10⁻⁴ s⁻¹, less than 5.0×10⁻⁴ s⁻¹, less than 6.0×10⁻⁴ s⁻¹, less than 7.0×10⁻⁴ s⁻¹, less than 8.0×10⁻⁴ s⁻¹, or less than 9.0×10⁻⁴ s⁻¹. In another embodiment, the binding affinity of a capture agent disclosed herein may have a disassociation rate constant of, e.g., more than 1×10⁻³ s⁻¹, more than 1×10⁻⁴ s⁻¹, or more than 1×10⁻⁵ s⁻¹. In other aspects of this embodiment, the binding affinity of a capture agent disclosed herein may have a disassociation rate constant of, e.g., more than 1.0×10⁻⁴ s⁻¹, more than 2.0×10⁻⁴ s⁻¹, more than 3.0×10⁻⁴ s⁻¹, more than 4.0×10⁻⁴ s⁻¹, more than 5.0×10⁻⁴ s⁻¹, more than 6.0×10⁻⁴ s⁻¹, more than 7.0×10⁻⁴ s⁻¹, more than 8.0×10⁻⁴ s⁻¹, or more than 9.0×10⁻⁴ s⁻¹.

In another embodiment, the binding affinity of a capture agent disclosed herein may have an equilibrium disassociation constant of less than or about 50 nM. In aspects of this embodiment, the binding affinity of a capture agent disclosed herein may have an equilibrium disassociation constant of, e.g., less than or about 50 nM, less than or about 20 nM, less than or about 10 nM, less than or about 5 nM, less than or about 2 nM, less than or about 1 nM, less than or about 0.5 nM, less than or about 0.45 nM, less than or about 0.4 nM, less than or about 0.35 nM, less than or about 0.3 nM, less than or about 0.25 nM, less than or about 0.2 nM, less than or about 0.15 nM, less than or about 0.1 nM, or less than or about 0.05 nM. In another embodiment, the binding affinity of a capture agent disclosed herein may have an equilibrium disassociation constant of more than 0.5 nM. In aspects of this embodiment, the binding affinity of a capture agent disclosed herein may have an equilibrium disassociation constant of, e.g., more than 0.5 nM, more than 0.45 nM, more than 0.4 nM, more than 0.35 nM, more than 0.3 nM, more than 0.25 nM, more than 0.2 nM, more than 0.15 nM, more than 0.1 nM, or more than 0.05 nM.

In yet another embodiment, the binding affinity of a capture agent disclosed herein may have an association rate constant for FVIII from an animal other than human of, e.g., less than 1×10⁰M⁻¹s⁻¹, less than 1×10¹M⁻¹s⁻¹, less than 1×10²M⁻¹s⁻¹, less than 1×10³M⁻¹s⁻¹, or less than 1×10⁴M⁻¹s⁻¹. In another embodiment, the binding affinity of a capture agent disclosed herein may have an association rate constant for FVIII from an animal other than human of, e.g., at most 1×10⁰M⁻¹s⁻¹, at most 1×10¹M⁻¹s⁻¹, at most 1×10²M⁻¹s⁻¹, at most 1×10³M⁻¹s⁻¹, or at most 1×10⁴M⁻¹s⁻¹.

Binding specificity is the ability of a capture agent to discriminate between a molecule containing its binding site or moiety and a molecule that does not contain that binding site or moiety. One way to measure binding specificity is to compare the Kon association rate of a capture agent for a molecule containing its binding site or moiety relative to the Kon association rate of a capture agent for a molecule that does not contain that binding site or moiety. For example, comparing the association rate constant (Ka) of a capture agent for human FVIII relative to an FVIII from other animals. In aspects of this embodiment, a capture agent that selectively binds to human FVIII has an association rate constant (Ka) for human FVIII that is, e.g., at least 2-fold more, at least 3-fold more, at least 4-fold more, at least 5-fold more, at least 6-fold more, at least 7-fold more, at least 8-fold more, or at least 9-fold more relative to an association rate constant (Ka) for an FVIII from other animals. In further aspects of this embodiment, a capture agent that selectively binds to human FVIII has an association rate constant (Ka) for human FVIII that is, e.g., at least 10-fold more, at least 100-fold more, at least 1,000-fold more or at least 10,000-fold more relative to an association rate constant (Ka) for an FVIII from other animals. In yet other aspects of this embodiment, a capture agent that selectively binds to human FVIII has an association rate constant (Ka) for human FVIII that is, e.g., at most 1-fold more, at most 2-fold more, at most 3-fold more, at most 4-fold more, at most 5-fold more, at most 6-fold more, at most 7-fold more, at most 8-fold more, or at most 9-fold more relative to an association rate constant (Ka) for an FVIII from other animals. In yet other aspects of this embodiment, a capture agent that selectively binds to human FVIII has an association rate constant (Ka) for human FVIII of that is, e.g., at most 10-fold more, at most 100-fold more, at most 1,000-fold more or at most 10,000-fold more relative to an association rate constant (Ka) for an FVIII from other animals.

The binding specificity of a capture agent can also be characterized as a binding specificity ratio of human FVIII relative to an FVIII from other animals. In aspects of this embodiment, a capture agent has a binding specificity ratio for human FVIII relative to an FVIII from other animals of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1. In yet other aspects of this embodiment, a capture agent has a binding specificity ratio for human FVIII relative to an FVIII from other animals of, e.g., at least 2:1, at least 3:1, at least 4:1, at least 5:1, at least 6:1, at least 7:1, at least 8:1, at least 9:1, at least 10:1, at least 15:1, at least 20:1, at least 25:1, at least 30:1, at least 35:1, or at least 40:1. In still other aspects of this embodiment, a capture agent has a binding specificity ratio for human FVIII relative to an FVIII from other animals of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1.

Binding avidity, also known as functional affinity, refers to the sum total of the functional binding strength between a multivalent capture agent and its binding site or moiety. A capture agent can have more than one binding site on human FVIII. While binding avidity of a capture agent depends on the binding affinities of the individual capture agent binding sites, binding avidity is greater than the binding affinity as all the agent-binding site interactions must be broken simultaneously for a capture agent to dissociate completely. It is envisioned that a capture agent can selectively bind to any and all binding sites or moieties for that capture agent.

Typically, the capture agent can distinguish human FVIII from an FVIII from other animals. FVIII from other animals may be present in a sample but will not selectively bind to a capture agent disclosed herein as the FVIII from other animals lacks the binding site that is required for capturing, or has much weaker binding affinity to the capture agent relative to human FVIII. One non-limiting example of FVIII from other animals is a naturally-occurring or endogenous FVIII expressed from the genome of the animal from which a sample was directly taken or derived from. Yet another non-limiting example of FVIII from other animals is FVIII expressed from cells of a cell culture line used to express human FVIII.

In some embodiments, a capture agent is an antibody, or an antibody fragment. An antibody refers to a molecule generated by an immune system that was made in response to a particular antigen that specifically binds to that antigen, and includes both naturally occurring antibodies and non-naturally occurring antibodies. An antibody can be a polyclonal antibody, a monoclonal antibody, a dimer, a multimer, a monospecific antibody, a bispecific antibody, such as, e.g., disulfide stabilized Fv fragments, scFv tandems [(scFv)2fragments], a multispecific antibody, a multivalent antibody, a humanized antibody, a camelized antibody, a chimeric antibody, bi-functional antibody, a cell-associated antibody like an Ig receptor, a linear antibody, a diabody, a tribody, a tetrabody, a minibody, or derivative or analog thereof, so long as the fragment exhibits the desired biological activity, and single chain derivatives of the same. An antibody can be a full-length immunoglobulin molecule comprising the V_{H} and V_{L} domains, as well as a light chain constant domain (C_{L}) and heavy chain constant domains, C_{H1}, C_{H2} and C_{H3}, or an immunologically active fragment of a full-length immunoglobulin molecule, such as, e.g., a Fab fragment, a F (ab′)₂fragment, a Fc fragment, a Fd fragment, a Fv fragment, a single chain Fv (scFv). An antibody can be derived from any vertebrate species (e.g., human, goat, horse, donkey, murine, rat, rabbit, or chicken), and can be of any type (e.g., IgG, IgE, IgM, IgD, and IgA), class (e.g., IgA, IgD, IgE, IgG, and IgM) or subclass (IgG1, IgG2, IgG3, IgG4, IgA1 and IgA2).

Antibodies useful to practice the methods disclosed herein are commercially available or can be generated according to methods that are well-known in the art. For example, monoclonal antibodies can be generated by the hybridoma method. Antibody fragments can be generated via proteolytic digestion of intact antibodies or can be produced directly by recombinant host cells. For example. Fab′ fragments can be directly recovered from E. coli and chemically coupled to form F(ab′)2 fragments. In another embodiment. F(ab′)2 can be formed using the leucine zipper GCN4 to promote assembly of the F(ab′)2 molecule. According to another approach, Fv, Fab or F(ab′)2 fragments can be isolated directly from recombinant host cell culture. Other techniques for the production of antibody fragments are apparent to a person with ordinary skill in the art.

In one embodiment, the capture antibody is GMA-8024, or an antigen-binding fragment or a variant thereof.

In one embodiment, the capture antibody is GMA-8023, or an antigen-binding fragment or a variant thereof.

In one embodiment, the capture antibody is GMA-012, or an antigen-binding fragment or a variant thereof.

The capture agent may take other forms (e.g., affibody, aptamer, synthetic peptide) as long as the capture agent is capable of specifically capturing human FVIII, and preferably not interfering with the activity assay, e.g., interfering with FVIII's role as cofactor for FIXa-mediated FXa generation. In some embodiments, the capture agent is an affibody. Affibody molecules are small highly robust proteins with specific affinities to target proteins. They are usually designed based on a three-helix bundle domain framework. In some embodiments, the capture agent is an aptamer. Aptamers are structured nucleic acid molecules that can bind to their targets with high affinity and specificity. Aptamers can assume a variety of shapes due to their tendency to form helices and single-stranded loops.

A capture agent disclosed herein may be attached to a solid phase as a support for the capture agent. As used herein, the term “solid-phase support” is synonymous with “solid support” or “solid phase” and refers to any matrix that can be used for immobilizing capture agent disclosed herein. A solid phase may be constructed using any suitable material with sufficient surface affinity to bind a capture agent. The solid-phase support selected can have a physical property that renders it readily separable from soluble or unbound material and generally allows unbound materials, such as, e.g., excess reagents, reaction by-products, or solvents, to be separated or otherwise removed (by, e.g., washing, filtration, centrifugation, etc.) from solid phase support-bound assay component. Non-limiting examples of how to make and use a solid phase supports are described in, e.g., Molecular Cloning, A Laboratory Manual, supra, (2001); and Current Protocols in Molecular Biology, supra, (2004), each of which is hereby incorporated by reference in its entirety.

Useful solid supports include: natural polymeric carbohydrates and their synthetically modified, crosslinked, or substituted derivatives, such as agar, agarose, cross-linked alginic acid, substituted and cross-linked guar gums, dextran, diazocellulose, carbohydrates, starch, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials can be used as filters with the above polymeric materials); and mixtures or copolymers of the above classes, such as graft copolymers obtained by initializing polymerization of synthetic polymers on a pre-existing natural polymer. Nitrocellulose and nylon can also be used. All of these materials can be used in suitable shapes, such as films, sheets, tubes, column; pins or “dipsticks”; a magnetic particle, particulates, microparticles, beads, or plates, or they can be coated onto, bonded, or laminated to appropriate inert carriers, such as paper, glass, plastic films, fabrics, or the like.

Alternatively, a solid support can constitute microparticles. Appropriate microparticles include those composed of polystyrene, polymethylacrylate, polypropylene, latex, polytetrafluoroethylene, polyacrylonitrile, polycarbonate, or similar materials. Further. the microparticles can be magnetic or paramagnetic microparticles, so as to facilitate manipulation of the microparticle within a magnetic field. Microparticles can be suspended in the mixture of soluble reagents and biological sample or can be retained and immobilized by a support material. In the latter case, the microparticles on or in the support material are not capable of substantial movement to positions elsewhere within the support material. Alternatively, the microparticles can be separated from suspension in the mixture of soluble reagents and biological sample by sedimentation or centrifugation. When the microparticles are magnetic or paramagnetic the microparticles can be separated from suspension in the mixture of soluble reagents and biological sample by a magnetic field.

A capture agent may be attached to the solid support by adsorption, where it is retained by hydrophobic forces. Alternatively, the surface of a solid support may be activated by chemical processes that cause covalent linkage of the capture agent to the support. A capture agent may be attached to the solid phase by ionic capture, where it is retained by a charged polymer.

After the incubation step disclosed herein, a washing step may be performed in order to remove any unbound components from the mixture, including unbound human FVIII, non-human FVIII and/or other agents that may interfere with the subsequence activity assay. Washing may be performed by washing the solid support containing the capture agent and captured human FVIII with a washing buffer. In some embodiments, the washing buffer comprises phosphate-buffered saline (PBS) and Tween (termed PBST buffer). In one embodiment, the washing buffer comprises PBS and 0.05% Tween 20. In another embodiment, the washing buffer comprises PBS and about 0.01%, 0.02%, 0.03%, 0.04%, 0.05%, 0.06%, 0.07%, 0.08%, 0.09% or 0.1% Tween 20. Washing may be performed one time, two times, three times, four times, five times, six times, seven times, eight times, nine times, ten times, or more than ten times. In one embodiment, washing is performed three times.

Detection of the presence or measurement of the activity of human FVIII disclosed herein can be accomplished by any assay that can qualitatively or quantitatively measure a characteristic indicative of the presence or an activity associated with human FVIII being monitored, including, without limitation, an in vitro assay, a cell-based assay, or an in vivo assay. The assay may be a non-specific polypeptide assay, such as, e.g., UV absorption assay or a chemical-based assay like a Bradford assay or a biuret assay, or a specific polypeptide assay, such as, e.g., a chromogenic assay, a colorimetirc assay, a chronometric assay, a chemiluminescense assay, an electrochemiluminescence assay, a bioluminescence assay, a fluorogenic assay, a resonance energy transfer assay, a plane polarization assay, a flow cytometry assay, an immuno-based assay or an activity assay like an enzymatic activity, an inhibitory activity, a coagulation activity, or a polymerization activity. The actual assay used to detect a characteristic of human FVIII or measure the human FVIII activity can be determined by a person of ordinary skill in the art by taking into account factors, including, without limitation, the amount of human FVIII present, the characteristic being assayed, and the preference of the person of ordinary skill in the art. Detecting the presence or an activity of human FVIII disclosed herein can be practiced in a singleplex or multiplex fashion.

Some embodiments of the method disclosed herein employ a chromogenic assay. A chromogenic assay typically uses peptide substrates that are composed of a specific oligopeptide or polypeptide moiety and a chromophore (dye carrier) and are customarily used for determining factors possessing protease activity, for example for determining coagulation factors (e.g., FVIII) in blood and plasma samples. The chromogenic peptide substrate, which is initially colorless, is cleaved, in dependence on the quantity and/or activity of human FVIII which is present in the sample, thereby releasing the chromophore. Cleavage changes the optical properties of the product, which are different from those of the uncleaved substrate and which can be measured by means of spectrophotometry. Non-limiting examples of chromogenic groups which can be coupled to a peptide substrate include para-nitroaniline (pNA), 5-amino-2-nitrobenzoic acid (ANBA), 7-amino-4-methoxycoumarin (ANC), quinony lamide (QUA), dimethyl 5-aminoisophthalate (DPA) and their derivatives. Fluorogenic substrates include, without limitation, Z-Gly-Pro-Arg-AMC [Z-Benzyloxycarbonyl; AMC=7-amino-4-methylcoumarin], homovanillic acid, 4-hydroxy-3-methoxyphenylacetic acid, reduced phenoxazines, reduced benzothiazines, Amplex®, resorufin β-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236, incorporated herein by reference in their entirety), 4-methylumbelliferyl β-D-galactopyranoside, carboxyumbelliferyl β-D-galactopyranoside and fluorinated coumarin β-D-galactopyranosides (U.S. Pat. No. 5,830,912, incorporated herein by reference in its entirety).

A non-limiting activity assay is a chromogenic assay used to measure FVIII activity based on the blood coagulation cascade. In this assay, thrombin activated Factor VIII forms a complex with Factor IXa, and this complex subsequently activates Factor X. Activated Factor X activity can be accessed by the hydrolysis of a chromogenic substrate which liberates a chromogenic group like p-nitro-aniline (pNA). The initial rate of pNA release, as determined by a change in absorbance per minute measured at 405 nm in dOD, is proportional to the Factor Xa activity and subsequently to the FVIII activity in the sample. By using excess of Factor IXa, and Factor X, the rate of activation of Factor X is solely proportional to the amount of thrombin cleaved Factor VIII present in the sample.

In some embodiments, the chromogenic assay can include one or more steps of: adding to the reaction mixture activated factor IX (FIXa), factor X (FX), calcium ion, phospholipids and thrombin and incubating the reaction mixture under conditions and for a time sufficient to generate activated FX (FXa); adding to the reaction mixture an FXa specific chromogenic substrate and incubating the reaction mixture under conditions and for a time sufficient to generate a measurable signal; optionally, adding to the reaction mixture a stopping agent to stop signal generation from the FXa specific chromogenic substrate; and measuring the signal generated from the reaction mixture.

The reagents of the chromogenic assay including activated factor IX (FIXa), factor X (FX), calcium ion, phospholipids and thrombin may be added in any sequence. In some embodiments, reagents of the chromogenic assay including activated factor IX (FIXa), factor X (FX), calcium ion, phospholipids and thrombin are added step-wise. For example, thrombin may be added first to allow FVIII to be activated to form activated FVIII (FVIIIa). Activated factor IX (FIXa) and factor X (FX) may be added subsequently and in excess to allow FVIIIa to form the tenase complex with FX and FIXa, resulting in the generation of activated factor X (FXa). In alternative embodiments, reagents of a chromogenic assay including activated factor IX (FIXa), factor X (FX), calcium ion, phospholipids and thrombin are added at the same time. In some embodiments, thrombin may be supplied as prothrombin. Reagents may be of any source, e.g., human or animal (mainly bovine source).

The FXa specific chromogenic substrate can be any substrate that has a selectivity for FXa and with a chromogenic group such as those described herein. In one embodiment. the chromogenic substrate is CH₃OCO-D-cyclohexylalanine-Gly-Arg-pNA. The chromogenic substrate solution may also contain a synthetic thrombin inhibitor.

After the FXa specific chromogenic substrate is added, the reaction may be followed by continuous reading of color development (absorbance increase) for a kinetic measurement or by stopping color development after a pre-defined time for an end-point measurement. For the latter, a stopping agent may be used.

In some embodiments, the stopping agent is acetic acid. In one embodiment, the stopping agent is about 20% acetic acid. In other embodiments, the stopping agent is about 5%, about 10%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40% acetic acid.

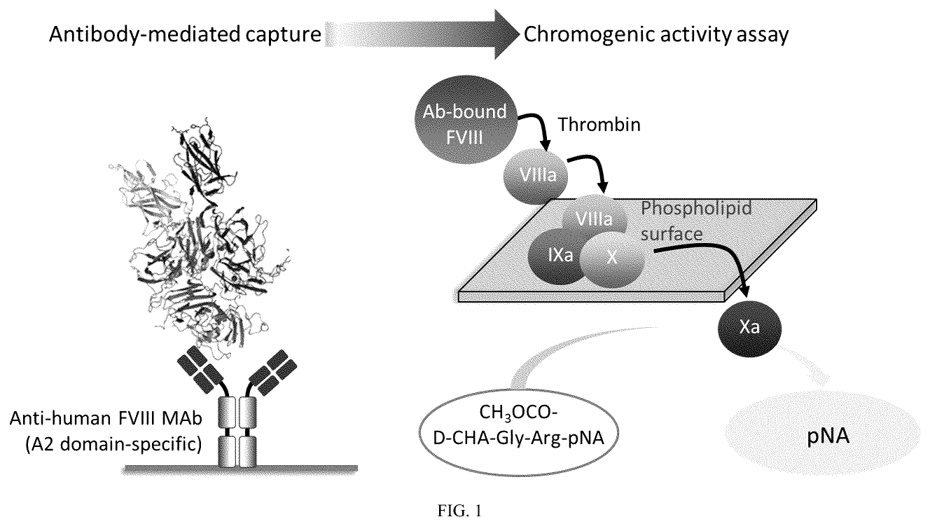

In a particular embodiment, the selective chromogenic FVIII activity assay is based on specifically capturing human FVIII by anti-human FVIII antibodies that are immobilized on the wells of a microplate (FIG. 1). After incubation, a subsequent washing step removes any unbound components, including animal FVIII. Then, reagents of a chromogenic assay for FVIII are added, which include activated FIXa (FIXa), factor X (FX), calcium, phospholipids and thrombin, allowing activated factor FVIII (FVIIIa) to act as a cofactor for FIXa-mediated FXa generation. Finally, the activated FX (FXa) generated during the assay is detected by a specific substrate for FXa. FVIII chromogenic activity is determined by comparison with a FVIII reference preparation.

Aspects of the present disclosure comprise, in part, a sample comprising human FVIII disclosed herein. A sample may be any material to be tested for the presence or activity of human FVIII disclosed herein. A variety of samples can be assayed according to a method disclosed herein including, without limitation, purified, partially purified, or unpurified human FVIII; a formulated human FVIII product; crude, fractionated or partially purified, or purified cell lysates from, e.g., bacteria, yeast, insect, or mammalian sources; and cell, tissue, or organ samples. A sample can be from any subject individual, including but not limited to, insects or mammals such as, e.g., human, bird, porcine, equine, bovine, murine, cat, rat, dog, or sheep. In some embodiments, the sample is obtained from a non-human animal. In some embodiments, the sample is obtained from a laboratory animal, including but not limited to, sheep, goat, cynomolgus monkey, rabbit, rat, rhesus macaque, mouse, swine, hamster, minipig, and guinea pig.

In one aspect of this embodiment, a sample may be a biological sample that contains or potentially contains human FVIII. A biological sample can include any cell, tissue, or organ sample taken directly from an individual. A biological sample can also be a sample of any bodily fluid taken directly from an individual including, without limitation, blood, urine, sputum, semen, feces, saliva, bile, cerebral fluid, nasal swab, urogenital swab, nasal aspirate, spinal fluid, etc. A biological sample can also include any preparation derived from a sample taken directly from an individual including, without limitation, a plasma fraction of a blood sample, a serum fraction of a blood sample, or an eluate from a purification process. A blood sample refers to any sample taken or derived from blood, such as a whole blood sample, a blood plasma sample or a blood serum sample. In some embodiments, the sample is a citrated plasma sample. In some embodiments, the sample may contain additives such as those used in the manufacturing of recombinant human FVIII. A non-limiting example of such additive is a solvent detergent (S/D) solution, often used as an effective virus inactivation solution, containing 1% Triton X-100, 0.3% Polysorbate 80 and 0.3% tri-n-butyl phosphate (tnBP). In some embodiments, the sample may contain one or more FVIII activity-mimicking agents. FVIII-activity mimicking agents are known to bias FVIII measurement with conventional methods. FVIII-activity mimicking agents include, but are not limited to, antibodies (e.g., bispecific antibody), antigen-binding fragments, peptides, nucleic acids, or small molecules. In one embodiment, the FVIII-activity mimicking agent is a bispecific antibody, such as Emicizumab.

It is envisioned that the method described herein makes it possible to selectively determine the activity of human FVIII administered to animals or added to animal plasma in addition to the animal's endogenous FVIII. The method described herein can specifically measure the procoagulant activity of investigational human FVIII products in non-clinical models, even in the presence of normal levels of endogenous animal FVIII. Currently available methods of measuring FVIII activity may not be capable of discriminating between human FVIII (whether added directly or generated by gene transfer) and endogenous animal FVIII, but rather measure the activity of both human and animal coagulation factors. Unfortunately, this measurement may introduce bias, becoming even higher at the end of the circulatory half-life of administered/generated human FVIII where the difference between administered/generated and endogenous FVIII reaches zero.

Furthermore, the method described herein can also allow the measurement of FVIII activity in sample matrices not currently available for analysis, e.g., due to interference. A non-limiting example of such an interfering sample matrix is the solvent-detergent (S/D) solution, used for the virus inactivation included in the manufacturing process of plasma-derived and recombinant FVIII. However, it is of interest to determine and monitor FVIII activity in as many intermediates and sample types as possible. The method described herein can also allow measurement in the presence of solvent-detergent solution and individual compounds.

A sample may be treated in a way to improve the detectability of human FVIII or its activity within the sample. Such treatments may, e.g., reduce the viscosity of the sample or purify a component fraction of the sample. Methods of treatment can involve lysing, dilution, purification, extraction, filtration, distillation, separation, concentration, inactivation of interfering components, and the addition of reagents. In addition, a solid material suspected of containing human FVIII may be used as a test sample once it is modified to form a liquid medium or to release human FVIII. The selection and pretreatment of biological samples prior to testing is well known in the art.

In some embodiments, the sample is diluted prior to the incubation step so that the FVIII activity can be measured with an acceptable level of accuracy and precision. In some embodiments, the sample is diluted by a factor of between 1:1 to about 1:100,000. In some embodiments, the sample is diluted by a factor of between 1:2 to about 1:20000. In some embodiments, the sample is diluted by a factor of about 1:5, about 1:10, about 1:20, about 1:40, about 1:80, about 1:100, about 1:160, about 1:200, about 1:320, about 1:400, about 1:500, about 1:640, about 1:800, about 1:1000, about 1:1280, about 1:1200, about 1:1500, about 1:1600, about 1:1800, about 1:2000, about 1:2400, about 1:2500, about 1:4000, about 1:5000, about 1:8000, about 1:10000, about 1:12000, about 1:14000, about 1:15000, about 1:16000, about 1:18000, or about 1:20000.

In some embodiments, the sample may be diluted with a dilution buffer compatible with preserving FVIII activity. An exemplary dilution buffer used herein comprises imidazole and NaCl. In some embodiments, the dilution buffer comprises about 1-10 g/L imidazole and about 1-10 g/L NaCl, and has a pH of between 7-8. In some embodiments, the dilution buffer comprises about 1, about 1.5, about 2, about 2.1, about 2.2, about 2.3, about 2.4, about 2.5, about 2.6, about 2.7, about 2.8, about 2.9, about 3, about 3.1. about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.5, about 6, about 6.5, about 7, about 7.5, about 8, about 8.5, about 9, about 9.5, about 10, about 1-3, about 2-4. about 3-5, about 2.5-4.5, about 3-6, about 4-8 or about 5-10 g/L imidazole. In some embodiments, the dilution buffer comprises about 1, about 1.5, about 2, about 2.5, about 3, about 3.5, about 4, about 4.1, about 4.2, about 4.3, about 4.4, about 4.5, about 4.6, about 4.7. about 4.8, about 4.9, about 5, about 5.1. about 5.2, about 5.3, about 5.4, about 5.5, about 5.6, about 5.7, about 5.75, about 5.8, about 5.85, about 5.9, about 5.95, about 6, about 6.1, about 6.3. about 6.4, about 6.5, about 6.6, about 6.7, about 6.8, about 6.9, about 7, about 7.1, about 7.2, about 7.3, about 7.4, about 7.5, about 8, about 8.5, about 9), about 9.5, about 10, about 1-3, about 2-4, about 3-5, about 2.5-4.5, about 3-6, about 4-8 or about 5-10 g/L NaCl. In some embodiments, the dilution buffer has a pH of about 7.0, about 7.1, about 7.2, about 7.3, about 7.4, about 7.5, about 7.6, about 7.7, about 7.8, about 7.9, or about 8.0. In one embodiment, the dilution buffer comprises about 3.4 g/L imidazole, about 5.85 g/L NaCl and has pH of about 7.4.

In some embodiments, incubation of the sample with the capture agent is performed under conditions allowing the selective binding of the capture agent to the human FVIII. In some embodiments, incubation of the sample with the capture agent is performed for about 10 minutes, about 20 minutes, about 30 minutes, about 40 minutes, about 50 minutes, about 1 hour, about 70 minutes about 80 minutes, about 90 minutes, about 100 minutes, about 110 minutes, about 2 hours or about 3 hours at room temperature. In some embodiments. incubation of the sample with the capture agent is performed for about 10 minutes, about 20 minutes, about 30 minutes, about 40 minutes, about 50 minutes, about 1 hour, about 70 minutes about 80 minutes, about 90 minutes, about 100 minutes, about 110 minutes, about 2 hour or about 3 hours at 37° C. In some embodiments, incubation of the sample with the capture agent is performed for about 10 minutes, about 20 minutes, about 30 minutes, about 40 minutes, about 50 minutes, about 1 hour, about 70 minutes about 80 minutes, about 90 minutes, about 100 minutes, about 110 minutes, about 2 hour or about 3 hours at 20° C.

In some embodiments, incubation of the sample with the capture agent is performed under conditions allowing the selective binding of the capture agent to the human FVIII. In some embodiments, incubation of the sample with the capture agent is performed for about 10 minutes, about 20 minutes, about 30 minutes, about 40 minutes, about 50 minutes, about 1 hour, about 70 minutes about 80 minutes, about 90 minutes, about 100 minutes, about 110 minutes, about 2 hours, about 3 hours, about 20-30 minutes, about 30-60 minutes, about 40-80 minutes, about 60-90 minutes, or about 1-2 hours at room temperature. In some embodiments, incubation of the sample with the capture agent is performed for about 10 minutes, about 20 minutes, about 30 minutes, about 40 minutes, about 50 minutes, about 1 hour, about 70 minutes about 80 minutes, about 90 minutes, about 100 minutes, about 110 minutes, about 2 hour, about 3 hours, about 20-30 minutes, about 30-60 minutes, about 40-80 minutes, about 60-90 minutes, or about 1-2 hours at about 37° C. In some embodiments. incubation of the sample with the capture agent is performed for about 10 minutes, about 20 minutes, about 30 minutes, about 40 minutes, about 50 minutes, about 1 hour, about 70 minutes about 80 minutes, about 90 minutes, about 100 minutes, about 110 minutes, about 2 hour, about 3 hours, about 20-30 minutes, about 30-60 minutes, about 40-80 minutes, about 60-90 minutes, or about 1-2 hours at about 20° C.

In some embodiments, incubation of the reaction mixture after addition of reagents of the chromogenic assay (e.g., activated factor IX (FIXa), factor X (FX), calcium ion, phospholipids and thrombin) is performed under conditions and for a time sufficient to generate activated FX (FXa). In some embodiments, incubation of the reaction mixture after addition of reagents of the chromogenic assay (e.g., activated factor IX (FIXa), factor X (FX), calcium ion, phospholipids and thrombin) is performed for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 25 minutes, about 30 minutes, about 40 minutes, about 50 minutes, about 1 hour, about 2 hours, about 1-10 minutes, about 5-12 minutes, about 8-15 minutes, about 10-20 minutes, about 15-30 minutes. or about 20-40 minutes at room temperature. In some embodiments, incubation of the reaction mixture after addition of reagents of the chromogenic assay (e.g., activated factor IX (FIXa), factor X (FX), calcium ion, phospholipids and thrombin) is performed for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 25 minutes, about 30 minutes, about 40 minutes, about 50 minutes, about 1 hour, about 2 hours, about 1-10 minutes, about 5-12 minutes, about 8-15 minutes, about 10-20 minutes, about 15-30 minutes, or about 20-40 minutes at about 37° C. In some embodiments, incubation of the reaction mixture after addition of reagents of the chromogenic assay (e.g., activated factor IX (FIXa), factor X (FX), calcium ion, phospholipids and thrombin) is performed for 1 minute, about 2 minutes, about 3 minutes, about 4 minutes, about 5 minutes, about 6 minutes, about 7 minutes, about 8 minutes, about 9 minutes, about 10) minutes, about 11 minutes, about 12 minutes, about 13 minutes, about 14 minutes, about 15 minutes, about 16 minutes, about 17 minutes, about 18 minutes, about 19 minutes, about 20 minutes, about 25 minutes, about 30 minutes, about 40 minutes, about 50 minutes, about 1 hour, about 2 hours, about 1-10 minutes, about 5-12 minutes, about 8-15 minutes, about 10-20 minutes, about 15-30 minutes, or about 20-40 minutes at about 20° C.

In some embodiments, a reaction buffer is provided for the chromogenic assay. An exemplary reaction buffer used herein comprises about 1-10 g/L Tris, about 0.1-10 g/L Na2EDTA, about 10-50 g/L NaCl, and has a pH 7.5-9. In some embodiments, the reaction buffer comprises about 1, about 2, about 3, about 3.5, about 4, about 4.5, about 5, about 5.1. about 5.2, about 5.3, about 5.4, about 5.5, about 5.6, about 5.7, about 5.8, about 5.85, about 5.9, about 5.95, about 6, about 6.06, about 6.1, about 6.15, about 6.2, about 6.25, about 6.3, about 6.35, about 6.4, about 6.45, about 6.5, about 6.55, about 6.6, about 6.65, about 6.7, about 6.75, about 6.8, about 6.85, about 6.9, about 7, about 7.1, about 7.2, about 7.3, about 7.4, about 7.5, about 7.6, about 7.7, about 7.8, about 7.9, about 8, about 9, about 10, or about 2-4, about 3-8, about 4-7, about 6-7, about 5-8, or about 6-9 g/L Tris. In some embodiments, the reaction buffer comprises about 0.1, about 0.5, about 1, about 2, about 2.1, about 2.2, about 2.3, about 2.4, about 2.5, about 2.6, about 2.7, about 2.75, about 2.8, about 2.85, about 2.9, about 2.95, about 3, about 3.03, about 3.05, about 3.1. about 3.15, about 3.2, about 3.25, about 3.3, about 3.35, about 3.4, about 3.45, about 3.5, about 3.55, about 3.6, about 3.65, about 3.7. about 3.75, about 3.8, about 3.85, about 3.9, about 4, about 4.1, about 4.2, about 4.3, about 4.4, about 4.5, about 5, about 6, about 7, about 8, about 9, about 10, about 0.1-4, about 1-5, about 2-4, about 3-4, about 3-5, about 4-6, or about 5-8 g/L Na₂EDTA. In some embodiments, the reaction buffer comprises about 10, about 15, about 16, about 17, about 18, about 19, about 20, about 21, about 22, about 23, about 24, about 25, about 26, about 27, about 28, about 29, about 30, about 31, about 32, about 33, about 34, about 35, about 37, about 40, about 45, about 50), about 10-20, about 15-25, about 20-40, about 20-30, or about 30-50 g/L NaCl. In some embodiments, the dilution buffer has a pH of about about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In one embodiment, the reaction buffer the comprises about 6.06 g/L Tris, about 3.03 g/L Na₂EDTA, about 25 g/L NaCl and has a pH of about 8.3.

In some embodiments, incubation of the reaction mixture after addition of the FXa specific chromogenic substrate (e.g., CH₃OCO-D-cyclohexylalanine-Gly-Arg-pNA) is performed under conditions and for a time sufficient to generate activated FX (FXa). In some embodiments, incubation of the reaction mixture after addition of the FXa specific chromogenic substrate is performed for about 5 minutes, about 6 minutes, about 7 minutes, about 8 minutes, about 9 minutes, about 10) minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30) minutes, about 40) minutes, about 50) minutes, about 1 hour, about 2 hours, about 1-10 minutes, about 5-15 minutes, about 10-20 minutes, about 15-30) minutes, about 20-40) minutes or about 25-50) minutes at room temperature. In some embodiments, incubation of the reaction mixture after addition of the FXa specific chromogenic substrate is performed for 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30) minutes, about 40) minutes, about 50) minutes, about 1 hour, about 2 hours, about 1-10 minutes, about 5-15 minutes, about 10-20) minutes, about 15-30 minutes, about 20-40 minutes or about 25-50) minutes at about 37° C. In some embodiments, incubation of the reaction mixture after addition of the FXa specific chromogenic substrate is performed for 5 minutes, about 6 minutes, about 7 minutes, about 8 minutes, about 9 minutes, about 10) minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30) minutes, about 40) minutes, about 50) minutes, about 1 hour, about 2 hours, about 1-10) minutes, about 5-15 minutes, about 10-20 minutes, about 15-30 minutes, about 20-40 minutes or about 25-50) minutes at about 20° C. A stopping agent (e.g., acetic acid) may be added to stop signal generation from the chromogenic substrate.

In various embodiments, the human FVIII being detected is a recombinant FVIII. In some embodiments, the human FVIII is full-length FVIII. In some embodiments, the human FVIII is B-domain deleted FVIII. In some embodiments, the human FVIII is a modified FVIII comprising one or more mutations as compared to a wild-type human FVIII. In some embodiments, the human FVIII is a modified B-domain deleted FVIII comprising one or more mutations as compared to a wild-type human FVIII in addition to the B-domain deletion. In some embodiments, the human FVIII is a recombinant FVIII fusion protein.

The methods disclosed herein may be evaluated by several parameters including. e.g., accuracy, precision, limit of detection (LOD), limits of quantitation (LOQ), range, specificity, selectivity, linearity, ruggedness, and system suitability. The accuracy of a method is the measure of exactness of an analytical method, or the closeness of agreement between the measured value and the value that is accepted as a conventional true value or an accepted reference value. The precision of a method is the degree of agreement among individual test results, when the procedure is applied repeatedly to multiple samplings of a homogeneous sample. As such, precision evaluates 1) within assay variability; 2) within-day variability (repeatability); and 3) between-day variability (intermediate precision); and 4) between-lab variability (reproducibility). Coefficient of variation (CV %) is a quantitative measure of precision expressed relative to the observed or theoretical mean value.

A method disclosed herein should be able to detect, over background, the presence or activity of human FVIII. The limit of detection (LOD) of a method refers to the concentration of human FVIII which gives rise to a signal that is significantly different from the negative control or blank and represents the lowest concentration of human FVIII that can be distinguished from background.

Thus, in an embodiment, a method disclosed herein can detect the LOD of human FVIII at an amount that is significantly different from a negative control or blank. In aspect of this embodiment, a method disclosed herein has an LOD of, e.g., 10 ng or less, 9 ng or less, 8 ng or less, 7 ng or less, 6 ng or less, 5 ng or less, 4 ng or less, 3 ng or less, 2 ng or less, 1 ng or less of human FVIII. In still other aspects of this embodiment, a method disclosed herein has an LOD of, e.g., 900 pg or less, 800 pg or less, 700 pg or less. 600 pg or less, 500 pg or less, 400 pg or less, 300 pg or less. 200 pg or less, 100 pg or less of human FVIII. In further aspects of this embodiment, a method disclosed herein has an LOD of, e.g., 90 pg or less, 80 pg or less; 70 pg or less, 60 pg or less, 50 pg or less, 40 pg or less, 30 pg or less, 20 pg or less, 10 pg or less of human FVIII. In other aspects of this embodiment, a method disclosed herein has an LOD of, e.g., 9 pg or less, 8 pg or less, 7 pg or less, 6 pg or less, 5 pg or less, 4 pg or less, 3 pg or less, 2 pg or less, 1 pg or less of human FVIII. In vet other aspects of this embodiment, a method disclosed herein has an LOD of, e.g., 0.9 pg or less, 0.8 pg or less, 0.7 pg or less, 0.6 pg or less, 0.5 pg or less, 0.4 pg or less, 0.3 pg or less, 0.2 pg or less, 0.1 pg or less of human FVIII.

In another aspect of this embodiment, a method disclosed herein has an LOD of, e.g., 10 nM or less or less, 9 nM or less, 8 nM or less, 7 nM or less, 6 nM or less, 5 nM or less, 4 nM or less, 3 nM or less, 2 nM or less, or I nM or less of human FVIII. In other aspects of this embodiment, a method disclosed herein has an LOD of, e.g., 900 pM or less, 800 pM or less, 700 pM or less, 600 pM or less, 500 pM or less, 400 pM or less, 300 pM or less, 200 pM or less, or 100 pM or less of human FVIII. In other aspects of this embodiment, a method disclosed herein has an LOD of, e.g., 100 pM or less, 90 pM or less, 80 pM or less, 70 pM or less, 60 pM or less, 50 pM or less, 40 pM or less, 30 pM or less, 20 pM or less, or 10 pM or less of human FVIII. In yet other aspects of this embodiment, a method disclosed herein has an LOD of, e.g., 10 pM or less of human FVIII, 9 pM or less, 8 pM or less, 7 pM or less, 6 pM or less. 5 pM or less. 4 pM or less. 3 pM or less. 2 pM or less, or 1 pM or less of human FVIII. In still other aspects of this embodiment, a method disclosed herein has an LOD of, e.g., 1000 fM or less, 900 fM or less, 800 fM or less, 700 fM or less, 600 fM or less, 500 fM or less, 400 fM or less, 300 fM or less, 200 fM or less, or 100 fM or less of human FVIII. In still other aspects of this embodiment, a method disclosed herein has an LOD of, e.g., 100 fM or less, 90 fM or less, 80 fM or less, 70 fM or less, 60 fM or less, 50 fM or less, 40 fM or less, 30 fM or less, 20 fM or less, or 10 fM or less of human FVIII. In still other aspects of this embodiment, a method disclosed herein has an LOD of, e.g., 10 fM or less, 9 fM or less, 8 fM or less, 7 fM or less, 6 fM or less, 5 fM or less, 4 fM or less, 3 fM or less, 2 fM or less, or 1 fM or less of human FVIII.

The limits of quantitation (LOQ) are the lowest and the highest concentrations of human FVIII in a sample or specimen that can be measured with an acceptable level of accuracy and precision. The lower limit of quantitation refers to the lowest dose that a detection method can measure consistently from the background. The upper limit of quantitation is the highest dose that a detection method can measure consistently before saturation of the signal occurs. The linear range of the method is the area between the lower and the upper limits of quantitation. The linear range is calculated by subtracting lower limit of quantitation from the upper limit of quantitation. As used herein, the term “signal to noise ratio for the lower asymptote” refers to the signal detected in the method at the lower limit of detection divided by the background signal. As used herein, the term “signal to noise ratio for the upper asymptote” refers to the signal detected in the method at the upper limit of detection divided by the background signal.

Thus, in an embodiment, a method disclosed herein can detect the LOQ of human FVIII at an amount that is significantly different from a negative control or blank. In aspect of this embodiment, a method disclosed herein has an LOQ of, e.g., 10 ng or less, 9 ng or less, 8 ng or less, 7 ng or less, 6 ng or less, 5 ng or less, 4 ng or less, 3 ng or less, 2 ng or less, 1 ng or less of human FVIII. In still other aspects of this embodiment, a method disclosed herein has an LOQ of, e.g., 900 pg or less, 800 pg or less, 700 pg or less, 600 pg or less, 500 pg or less, 400 pg or less, 300 pg or less, 200 pg or less, 100 pg or less of human FVIII. In further aspects of this embodiment, a method disclosed herein has an LOQ of, e.g., 90 pg or less, 80 pg or less, 70 pg or less, 60 pg or less, 50 pg or less, 40 pg or less, 30 pg or less, 20 pg or less, 10 pg or less of human FVIII. In other aspects of this embodiment, a method disclosed herein has an LOQ of, e.g., 9 pg or less, 8 pg or less, 7 pg or less, 6 pg or less, 5 pg or less, 4 pg or less, 3 pg or less, 2 pg or less, 1 pg or less of human FVIII. In yet other aspects of this embodiment, a method disclosed herein has an LOQ of, e.g., 0.9 pg or less, 0.8 pg or less, 0.7 pg or less, 0.6 pg or less, 0.5 pg or less, 0.4 pg or less, 0.3 pg or less, 0.2 pg or less, 0.1 pg or less of human FVIII.

In another aspect of this embodiment, a method disclosed herein has an LOQ of, e.g., 10 nM or less, 9 nM or less, 8 nM or less, 7 nM or less, 6 nM or less, 5 nM or less, 4 nM or less, 3 nM or less, 2 nM or less, or 1 nM or less of human FVIII. In other aspects of this embodiment, a method disclosed herein has an LOQ of, e.g., 900 pM or less, 800 pM or less, 700 pM or less, 600 pM or less, 500 pM or less, 400 pM or less, 300 pM or less, 200 pM or less, or 100 pM or less of human FVIII. In other aspects of this embodiment, a method disclosed herein has an LOQ of, e.g., 100 pM or less, 90 pM or less, 80 pM or less, 70 pM or less, 60 pM or less, 50 pM or less, 40 pM or less, 30 pM or less, 20 pM or less, or 10 pM or less of human FVIII. In yet other aspects of this embodiment, a method disclosed herein has an LOQ of, e.g., 10 pM or less of human FVIII, 9 pM or less, 8 pM or less, 7 pM or less, 6 pM or less, 5 pM or less, 4 pM or less, 3 pM or less, 2 pM or less, or 1 pM or less of human FVIII. In still other aspects of this embodiment, a method disclosed herein has an LOQ of, e.g., 1000 fM or less, 900 fM or less, 800 fM or less, 700 fM or less, 600 fM or less, 500 fM or less, 400 fM or less, 300 fM or less, 200 fM or less, or 100 fM or less of human FVIII. In still other aspects of this embodiment, a method disclosed herein has an LOQ of, e.g., 100 fM or less, 90 fM or less, 80 fM or less, 70 fM or less, 60 fM or less, 50 fM or less, 40 fM or less, 30 fM or less, 20 fM or less, or 10 fM or less of human FVIII. In still other aspects of this embodiment, a method disclosed herein has an LOQ of, e.g., 10 fM or less, 9 fM or less, 8 fM or less, 7 fM or less, 6 fM or less, 5 fM or less, 4 fM or less, 3 fM or less, 2 fM or less, or 1 fM or less of human FVIII.

A method disclosed herein may have a precision of no more than 50%. In aspects of this embodiment, a method disclosed herein may have a precision of no more than 50%, no more than 40%, no more than 30%, or no more than 20%. In other aspects of this embodiment, a method disclosed herein has a precision of no more than 15%, no more than 10%, or no more than 5%. In other aspects of this embodiment, a method disclosed herein may have a precision of no more than 4%, no more than 3%, no more than 2%, or no more than 1%.

An method disclosed herein may have an accuracy of at least 50%. In aspects of this embodiment, a method disclosed herein may have an accuracy of at least 50%, at least 60%, at least 70%, or at least 80%. In other aspects of this embodiment, a method disclosed herein may have an accuracy of at least 85%, at least 90%, or at least 95%. In other aspects of this embodiment, a method disclosed herein may have an accuracy of at least 96%, at least 97%. at least 98%, or at least 99%.

A method disclosed herein may have a signal to noise ratio for the lower asymptote that is statistically significant and a signal to noise ratio for the upper asymptote that is statistically significant. In aspects of this embodiment, a method disclosed herein may have a signal to noise ratio for the lower asymptote of, e.g., at least 3:1, at least 4:1, at least 5:1, at least 6:1, at least 7:1, at least 8:1, at least 9:1, at least 10:1, at least 15:1 or at least 20:1. In other aspects of this embodiment, a method disclosed herein may have a signal to noise ratio for the upper asymptote of, e.g., at least 10:1, at least 15:1, at least 20:1, at least 25:1, at least 30:1, at least 35:1, at least 40:1, at least 45:1, at least 50:1, at least 60:1, at least 70:1, at least 80:1, at least 90:1, or at least 100:1, at least 150:1, at least 200:1, at least 250:1, at least 300:1, at least 350:1, at least 400:1, at least 450:1, at least 500:1, at least 550:1, or at least 600:1.

The specificity of a method disclosed herein defines the ability of the method to measure human FVIII to the exclusion of other relevant components, such as, e.g., FVIII from other animals. The selectivity of a method disclosed herein describes the ability of the method to differentiate various substances in a sample. The linearity of a method disclosed herein describes the ability of the method to elicit results that are directly, or by a well-defined mathematical transformation, proportional to the concentration of human FVIII in the sample. Thus in an embodiment, a method disclosed herein may distinguish human FVIII from FVIII from other animals having, e.g., 70 or less, 60% or less, 50% or less, 40% or less, 30% or less, 20% or less, or 10% or less the activity of FVIII from other animals.

The ruggedness of a method disclosed herein describes the reproducibility of the results obtained for identical samples under normal (but variable) conditions of the method. Robustness of a method disclosed herein describes the ability of the method to measure of its capacity to remain unaffected by small but deliberate variations in the method parameters and provides an indication of its reliability in normal usage. Thus, whereas ruggedness evaluates unavoidable changes, robustness evaluates deliberate changes. Typical parameters evaluated by ruggedness and robustness include the effects of freeze/thaw, incubation times, incubation temperature, longevity of reagent, sample preparation, sample storage, cell passage number, lots of toxin, variability between purifications, and variability between nicking reactions. Robustness parameters for a method disclosed herein include the cell bank (beginning, middle and end of freeze), cell passage level, cell seeding density, cell stock density (how many days in culture), cell age in flask (waiting time to seeding), incubation time, different plates, excessive amounts of serum, and source of reagents. The system suitability of a method disclosed herein describes the ability of the method to determine method performance, including the performance of reagents and instruments, over time by analysis of a reference standard. System suitability refers to the fact that equipment, electronics, assay performance, and samples to be analyzed, constitute an integrated system. System suitability can be evaluated by testing for parallelism, which is when plotting the log dose versus the response, serial dilutions of the reference and serial dilutions of the samples should give rise to parallel curves.

Aspects of the present specification disclose kits comprising one or more components useful for practicing the methods disclosed herein. The one or more components of a kit may comprise one or more capture agents disclosed herein, one or more solid phase supports, and/or one or more reagents necessary to detect the presence and/or measure an activity of human FVIII. A kit disclosed herein can include a solid phase and a capture agent affixed to the solid phase. A kit disclosed herein can include a one or more reagents used in the chromogenic assay including activated factor IX (FIXa), factor X (FX), calcium ion, phospholipids and thrombin, in one or separate vials. In some embodiments, thrombin may be supplied as prothrombin. In some embodiments, FIXa and FX may be combined in one vial, with phospholipids in a separate vial and calcium ion in a yet separate vial. In some embodiments, FIXa, phospholipids and Ca are combined in one vial and FX in a separate vial. A kit disclosed herein can also include an FXa specific chromogenic substrate (e.g., CH₃CO-D-cyclohexylalanine-Gly-Arg-pNA) and optionally a stopping agent (e.g., acetic acid). The chromogenic substrate vial may also contain a synthetic thrombin inhibitor. Reagents may be of any source, e.g., human or animal (mainly bovine source).

A kit disclosed herein may also comprise FVIII reference preparation useful as a positive control for capture agent and/or the assaying step. If desired, this component can be included in the test kit in multiple concentrations to facilitate the generation of a standard curve to which the signal detected in the test sample can be compared.

A kit disclosed herein can further include one or more of a binding buffer, a reaction buffer, an FVIII dilution buffer, and a washing buffer such as those described herein.

A kit generally includes a package with one or more containers holding the components, as one or more separate compositions or, optionally, as admixture where the compatibility of the reagents will allow. A kit can also include other material(s), which can be desirable from a user standpoint, such as a buffer(s), a diluent(s), a standard(s), and/or any other material useful in sample processing, washing, or conducting any other step of the assay. A kit disclosed herein may also include instructions for carrying out one or more methods disclosed herein. Instructions included in kits disclosed herein can be affixed to packaging material or can be included as a package insert. While the instructions are typically written or printed materials, they are not limited to such. Any medium capable of storing such instructions and communicating them to an end user is contemplated by the embodiments disclosed herein. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Description of the Monoclonal Antibody-Based Chromogenic FVIII Activity Assay

An ELISA reader ELx808 (BioTek; Szabo, Vienna, Austria), plate washers (ELx405, BioTek), a pipetting robotic system (Precision 2000; BioTek), and a plate shaker (PHMP-4; Grant Bio, Szabo, Vienna, Austria) were used to develop the method. Furthermore, 96-well Nunc MaxiSorp F96 flat-bottom plates were obtained from ThermoFisher Scientific (Vienna, Austria). The buffer chemicals were purchased from VWR (Vienna, Austria). Tween 20 (EIA grade) was obtained from Bio-Rad (Vienna, Austria), and benzamidine hydrochloride monohydrate was obtained from Sigma (Vienna, Austria). The monoclonal anti-factor VIII (FVIII) antibody was purchased from Green Mountain Antibodies (A2-HC; GMA-8024; Green Mountain Antibodies. Szabo, Vienna, Austria). The reagents for the chromogenic FVIII activity test (reagent A: phospholipid, human serum albumin; lot code #7M . . . ; reagent B: FIX, FX, Ca²⁺ human serum albumin, lot code #7L . . . ; substrate FXa-1: 10 μmol+0.012 μmol a-NAPAP/vial, lot code #8M . . . ; FVIII reaction buffer, 6.06 g/L Tris, 3.03 g/L Na2EDTA. 25 g/L NaCl, pH 8.3. lot code #9T . . . ; and FVIII dilution buffer 3.4 g/L imidazole. 5.85 g/L NaCl, pH 7.4. lot code #9Y . . . ) were from Baxter (Vienna, Austria). The following buffers/solutions were prepared: Coating buffer (0.1 M NaHCO₃, 0.1 M Na₂CO₃, pH 9.5), phosphate-buffered saline (PBS; 0.8% NaCl, 0.02% KCl, 0.02% KH₂PO₄, 0.126% Na₂HPO₄×2 H₂O, pH 7.2l), washing buffer (PBST; PBS with 0.05% Tween 20), blocking/dilution buffer (PBST with 0.1% skimmed dry milk, 2 mM benzamidine, 650 mM NaCl), stopping solution (20% acetic acid), and antibody capturing solution (anti-FVIII monoclonal antibody, diluted 1:100 in coating buffer).

100 μL/well antibody-capturing solution was coated at +2 to +10° C. overnight. After a washing step with PBST, done three times, the plate was blocked with 200 μL/well blocking buffer at room temperature (RT) for 1 h and then was washed. The wells were filled with 100 μL/well dilution buffer. Six serial 1+1 dilutions were prepared in duplicates directly on the plate for standard, samples and assay control. The positions B11 and B12 were used as assay blanks. The human plasma standard ORKL17 (Siemens) with a FVIII concentration of 0.97 IU/mL was used as the reference standard. The six-point calibration curve covered a FVIII activity range from 3.03 to 97 mIU/mL. Samples were diluted according to their estimated FVIII levels to obtain FVIII concentrations within the range of the calibration curve, while the assay control (Normal Reference Plasma CRYOcheck. Precision BioLogic CCNRP-05) was diluted 1/5 before the serial 1+1 dilutions were prepared on the plate. The dilutions were incubated for 1 h at RT. The washed plate was then incubated with FVIII dilution buffer (200 μL/well) for 10 min at RT and emptied, before the chromogenic FVIII activity assay was done using the Precision 2000 system. FVIII dilution buffer was mixed 1+1 with reagent A and loaded (40 μL/well); reagent B (20 μL/well) was added and the plate was incubated on the plate shaker at RT for 15 min. Substrate (100 μL/well) was added and after incubation at RT for 25 min, stopping solution was added (40 μL/well). The plate was measured at 405 nm (reference wavelength 620 nm); quantitative evaluation was based on a double-logarithmic calibration curve between the blank-corrected optical densities (ODs) and the FVIII concentrations of the six non-zero assay calibrators.

The assay (FIG. 1) included the specific capture step of human FVIII followed by the chromogenic activity assay, carried out with the human FVIII captured and immobilized on the surface of the wells. In this assay, FVIII was first activated to form activated FVIII (FVIIIa) by human thrombin. FVIIIa then formed the tenase complex with factor X (FX) and activated factor FIX (FIXa), supplied by the reagents in excess, resulting in the generation of activated factor X (FXa). The resulting FXa concentration, being within a defined range strictly dependent on the FVIII concentration, was then determined by the hydrolysis of the substrate CH₃OCO-D-cyclohexylalanine-Gly-Arg-pNA to form the chromogenic pNA.

### Example 2: Selection of an Appropriate Capturing Antibody

An anti-FVIII antibody suitable for use in the invented method should have at least the following features: (1) absolute selectivity for human FVIII and (2) the antibody's binding of FVIII should not interfere with the chromogenic FVIII activity test. The latter requirement was investigated for the anti-FVIII antibodies summarized in Table 1.

TABLE 1
Suitability investigation of anti-FVIII antibodies

| Name | Source | Epitope |
|---|---|---|
| GMA-8024 | Green Mountain Antibodies | Heavy chain, A2 domain |
| GMA-8023 | Green Mountain Antibodies | Heavy chain, A2 domain |
| ESH-4 | American Diagnostica | N/A |
| RFF-VIIIC/5 [1] | BioRad | Light chain |
| RFF-VIIIC/8 [2] | BioRad | Heavy chain |
| ESH-8 | American Diagnostica | N/A |
| GMA-012 | Green Mountain Antibodies | Heavy chain, A2 domain |
| N55195M | American Diagnostica | Light chain |

NB: Data on epitope specificity are as provided by the antibodies' manufacturers.

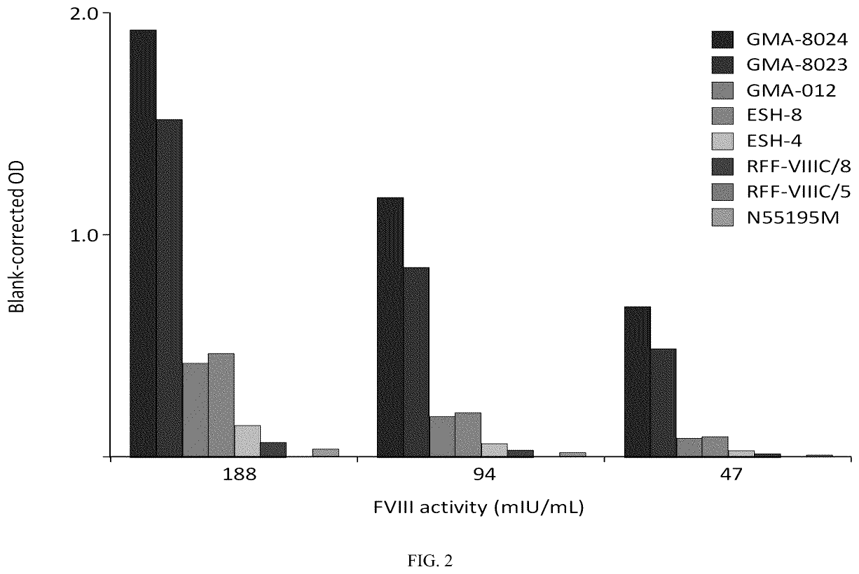

The antibodies were coated on the wells of a F96 microplate (100 μL/well) at a concentration of 10 μg/mL, achieved by dilution in PBS. After a washing step with PBST, serial 1+1 dilution series of B-domain deleted (BDD) rFVIII, prepared in PBST containing 650 mM NaCl, 1 mg/mL skimmed dry milk and 2 mM benzamidine, were loaded to the wells (100 μL/well) and incubated at RT for 60 min. The plate was then washed and further processed as described above in Example 1. FIG. 2 shows the blank-corrected ODs obtained for the BDD FFVIII concentrations of 188, 94 and 47 mU/mL for the anti-FVIII antibodies listed in Table 1. From left to right in FIG. 2, the anti-FVIII capturing antibodies are: GMA-8024, GMA-8023, GMA-012, ESH-8, ESH-4, RFF-VIIIC/8, RFF-VIIIC/5, and N55195M.

The responses in the chromogenic FVIII assay differed considerably, depending on the antibody used for capturing BDD FFVIII. Nevertheless, dose-dependent responses were observed for all those antibodies, which provided clear signals. Antibody GMA-8024, directed against the A2 domain of the heavy chain of FVIII, provided the highest signals for all three FVIII concentrations evaluated and was therefore used in the following Examples.

### Example 3: Assay Calibration Curves Obtained with a Human Reference Plasma Preparation

The human reference plasma preparation ORKL17 (Siemens) with a labelled FVIII activity of 0.97 IU/mL (lot No. 503267) was used for the assay calibration curve. This curve comprised six serial dilutions ( 1/10, 1/20, 1/40, 1/80, 1/160 and 1/320) and ranged from 3.03 to 97.0 mIU/mL. Table 2 shows the mean blank-corrected optical densities (ODs) for the six assay calibrators D1 to D6 and the blanks. Table 2 also shows the attributes of the log-log regression curves, calculated between the logarithms of the blank-corrected ODs and the FVIII concentrations of the assay standards D1 to D6. In particular, slopes, y-intercepts (y-int), correlation coefficients r and the relative total errors (RTEs) are shown. RTEs were calculated according to the formula RTE=|xₙ−x_{c}+2SD x_{c}×100, where xₙ and x_{c} represent the nominal and the calculated concentration of the assay standard, respectively. The assay standard's calculated concentration xₑ was obtained by inserting the blank-corrected ODs of the calibration curve standards in the equation of the calibration curve. The resulting concentration was multiplied with the respective dilution factor and finally, the six results were averaged, calculating also the standard deviation (SD x_{c}) of this mean. For these calibration curve attributes, the minimum and maximum values of 108 curves as well as mean and corresponding relative standard deviations are shown.

TABLE 2
Calibration curves with a human reference plasma preparation

| Attribute | Blank-corrected ODs | | | | | | OD | | | | RTE |
|---|---|---|---|---|---|---|---|---|---|---|---|
| (n = 108) | D1 | D2 | D3 | D4 | D5 | D6 | Blank | Slope | y-int | r | (%) |
| Minimum | 1.294 | 0.635 | 0.322 | 0.158 | 0.078 | 0.027 | 0.140 | 0.8489 | −2.0824 | 0.9977 | 1.6 |
| Maximum | 1.802 | 0.964 | 0.522 | 0.277 | 0.152 | 0.092 | 0.185 | 1.1725 | −1.4703 | 1.0000 | 18.3 |
| Mean | 1.531 | 0.806 | 0.429 | 0.225 | 0.118 | 0.066 | 0.153 | 0.9153 | −1.6382 | 0.9996 | 6.5 |
| RSD | 6.6 | 8.6 | 10.2 | 11.2 | 12.6 | 15.2 | 4.3 | 4.6 | −5.3 | N/A | N/A |

The abbreviations RSD and N/A stand for relative standard deviation and not applicable, respectively.

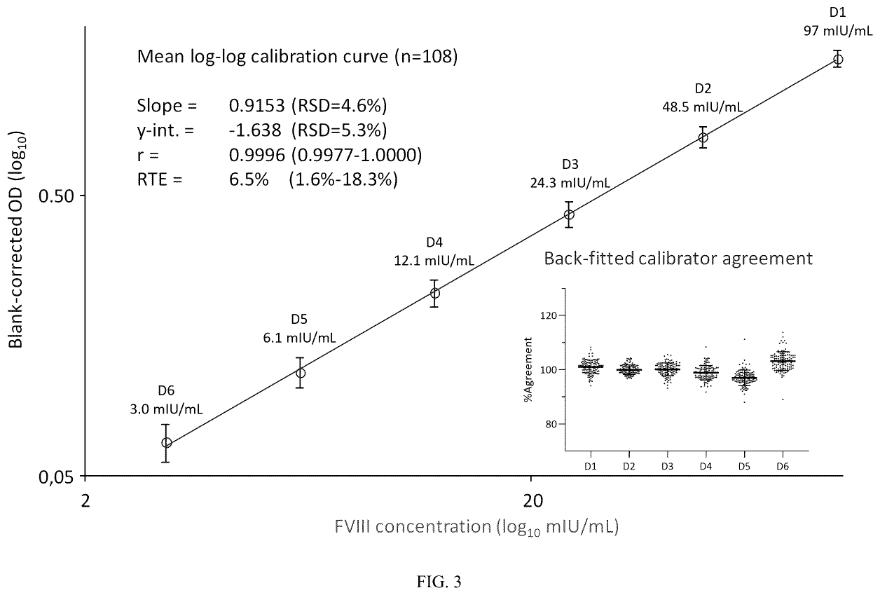

The 108 log-log calibration curves, ranging from 3.03 to 97.0 mIU FVIII/mL, demonstrated similar shape, good accuracy and adequate precision. Thus, the RSDs of the mean blank-corrected ODs determined for the assay standards DI to D6 had RSDs did not exceed 15.2% with higher RSDs shown for the lower concentrations. The RSDs for mean slope and mean y-intercept of the log-log calibration curves were 4.3% and −5.3%, respectively, demonstrating the similar shapes of the calibration curves. The mean correlation coefficient of 0.9996 with individual values ranging from 0.9977 to 1.0000, and the low mean RTE of 6.5%, with individual values ranging from 1.6% to 18.3%, both confirmed the accuracy of the curve fitting model applied. Of note, the RTE should not exceed 25% to indicate the adequate fitting of a given calibration curve. FIG. 3 shows the mean calibration curve and the agreement of the back-fitted assay calibrations to their nominal values.

The results of the calibrator back-fitting corroborated the good accuracy of the calibration curves as all back-fitted values deviated less than ±14% from their respective nominal values. This is clearly within the ±20% range defined, e.g., by the EMA guideline for the validation of bioanalytical methods [2]. The assay's sensitivity, allowing the accurate and precise determination of 3.03 mIU FVIII/mL, D6, was similar to that of the conventional chromogenic assay and seemed not to be obviously reduced, though FVIII had to be captured by the antibody before running the chromogenic test.

### Example 4: Assay Calibration Curves Obtained with Human B-Domain Deleted (BDD) Recombinant FVIII

Calibration curves were constructed using a human recombinant BDD FVIII preparation with an activity of 750 IU/mL using the method described in Example 1. The calibration curves ranged from 2.9 to 93.8 mIU/mL. Table 3 shows the mean blank-corrected ODs for the assay calibrators D1 to D6, the blanks and slopes, y-intercepts, correlation coefficients r and RTEs, calculated for the four log-log calibration curves.

TABLE 3
Calibration curves with recombinant BDD-FVIII preparations.

| Assay calibrators | | Mean blank-corrected ODs | | | | | |
|---|---|---|---|---|---|---|---|
| Name | mU/mL | AE-2911 | AE-2912 | AE-2913 | AE-2914 | Mean | RSD |
| D1 | 93.8 | 1.761 | 1.880 | 1.753 | 1.752 | 1.786 | 3.5 |
| D2 | 46.9 | 0.941 | 0.969 | 0.958 | 0.943 | 0.952 | 1.4 |
| D3 | 23.4 | 0.504 | 0.538 | 0.507 | 0.505 | 0.513 | 3.2 |
| D4 | 11.7 | 0.243 | 0.277 | 0.246 | 0.247 | 0.253 | 6.2 |
| D5 | 5.9 | 0.114 | 0.114 | 0.115 | 0.119 | 0.115 | 2.1 |
| D6 | 2.9 | 0.053 | 0.057 | 0.058 | 0.063 | 0.058 | 7.1 |
| Blank | 0.0 | 0.195 | 0.188 | 0.198 | 0.199 | 0.195 | 2.5 |

| | | | | | | |
|---|---|---|---|---|---|---|
| Slope | 1.0151 | 1.0125 | 0.9949 | 0.9723 | 0.9987 | 2.0 |
| y-intercept | −1.72 | −1.69 | −1.69 | −1.65 | −1.69 | −1.8 |
| r | 0.9989 | 0.9980 | 0.9992 | 0.9995 | 0.9989 | N/A |
| RTE | 12.5 | 17.0 | 10.6 | 8.0 | 12.0 | N/A |

The abbreviations RSD and N/A stands for relative standard deviation and not applicable, respectively.

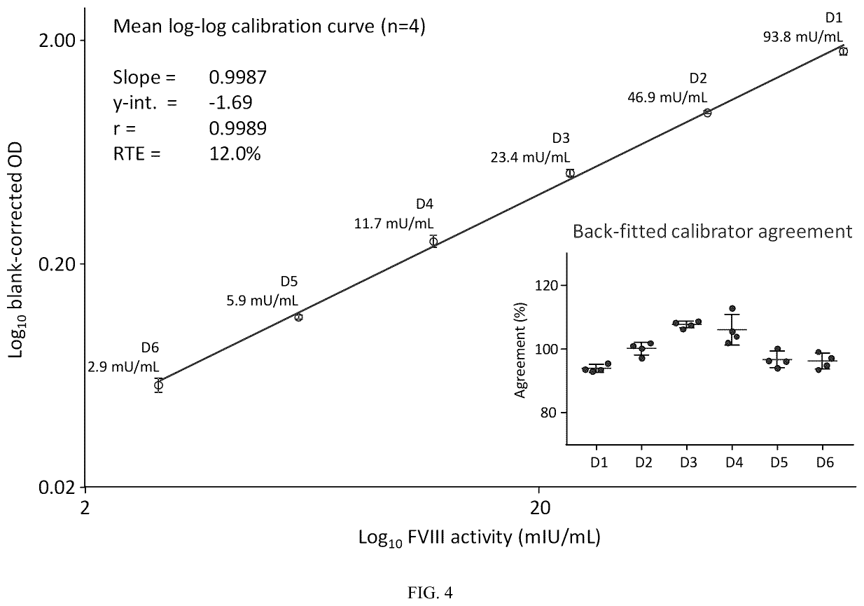

Given the location of the epitope of the capturing antibody GMA-8024, which is located in the A2 domain of the heavy chain, the signals obtained with the BDD FVIII preparation were similar in height to those of full-length plasma FVIII. The four (4) log-log calibration curves demonstrated similar shape, good accuracy and adequate precision. Thus, the RSDs determined for the mean blank-corrected ODs did not exceed 7.1%, while the RSDs for slope and y-intercept were as low as 2.0% and 1.8%, respectively. The mean correlation coefficient of 0.9989 and the mean RTE of 12.0% confirmed the accuracy of the curve fitting model applied. The adequate accuracy of the calibration curve fitting was corroborated by the results of the calibrator back-fitting, where all back-fitted values deviated less than +13% from their respective nominal values. The sensitivity of the assay, allowing the accurate and precise determination of 2.9 mU FVIII/mL as contained in the assay standard D6, was similar to that of a conventional chromogenic assay despite the additional step of capturing FVIII by the monoclonal antibody before running the chromogenic test. Furthermore, slopes in the same order of magnitude were obtained for plasma-derived full length FVIII and BDD FFVIII, evidencing that the capture step worked efficiently for both FVIII molecules. FIG. 4 shows the mean calibration curve obtained by using the BDD rFVIII preparation and the agreement of the back-fitted assay calibrations to their nominal values.

### Example 5: Selectivity Investigation for Laboratory Animal Citrated Plasma Samples

The selectivity of the antibody-based chromogenic FVIII activity assay was confirmed by the measurement of different citrated laboratory animal plasma samples at the minimum dilution of 1/10. Table 4 shows the results obtained providing the mean ODs of the assay standard D6 with the lowest FVIII concentration of 2.9 mU/mL, the mean ODs for the blanks and those measured for the 1/10-diluted animal plasma samples. In addition, the columns “Δblank %” and “ΔD6 %” relate the signals determined for the 1/10-diluted plasma samples with those of the blank and the assay standard D6, showing the relative differences calculated as (OD_{Plasma}−OD_{Blank})/OD_{Blank6}×100 and (OD_{Plasma}−OD_{D6})/OD_{D6}×100, respectively.

TABLE 4
Selectivity investigation for citrated animal plasma species

| | OD | | | Relative differences | | |
|---|---|---|---|---|---|---|
| Plasma | D6 | Blank | Plasma | ΔBlank % | ΔD6% | Test No. |
| Sheep | 0.248 | 0.195 | 0.196 | 0.5 | −21.0 | AE-2911 |
| Goat | 0.248 | 0.195 | 0.181 | −7.2 | −27.0 | AE-2911 |
| Cynomolgus | 0.248 | 0.195 | 0.175 | −10.3 | −29.4 | AE-2911 |
| Rabbit | 0.245 | 0.188 | 0.193 | 2.7 | −21.2 | AE-2912 |
| Rat | 0.245 | 0.188 | 0.184 | −2.1 | −24.9 | AE-2912 |
| Rhesus | 0.245 | 0.199 | 0.195 | −2.0 | −20.4 | AE-2914 |

Among the six (6) citrated animal plasma samples, only the sheep plasma sample showed a signal slightly higher than that of the assay blank, while all others provided slightly lower signals than the blank. These slight differences, however, did not indicate a substantial interference with the assay, but rather reflected measurement variability. Expressed as a percent of the lowest assay standard containing 2.9 mU FVIII/mL, the signals elicited by the 1/10-diluted animal plasma samples were at least 20.4% lower. These data confirmed the selectivity of the antibody-based chromogenic FVIII assay for human FVIII, as there was no evidence that the endogenous animal FVIII interfered with the assay.

### Example 6: Selectivity Investigation for Laboratory Animal Serum Samples

The selectivity of the antibody-based chromogenic FVIII activity assay was confirmed by the measurement of different animal serum samples at the minimum sample dilution of 1/10. Table 5 shows the results obtained providing the mean ODs of the assay standard D6 with the lowest FVIII concentration of 2.9 mU/mL, the mean ODs for the blanks and those measured for the 1/10-diluted animal serum samples. In addition, the columns “Δblank %” and “ΔD6 %” relate the signals determined for the serum samples with those of the blank and the assay standard D6, showing the relative differences calculated according to (OD_{Plasma}−OD_{Blank})/OD_{Blank}×100 and (OD_{Plasma}−OD_{D6})/OD_{D6}×100, respectively.

TABLE 5
Selectivity investigation for citrated animal serum species

| | OD | | | Relative differences | | |
|---|---|---|---|---|---|---|
| Serum | D6 | Blank | Serum | ΔBlank % | ΔD6% | Test No. |
| Mouse | 0.245 | 0.188 | 0.188 | 0.0 | −23.3 | AE-2912 |
| Swine | 0.256 | 0.198 | 0.196 | −1.0 | −23.4 | AE-2913 |
| Hamster | 0.256 | 0.198 | 0.193 | −2.5 | −24.6 | AE-2913 |
| Guinea pig | 0.261 | 0.199 | 0.196 | −1.5 | −24.9 | AE-2914 |

All serum samples provided slightly lower signals than the blank with the difference to the blank not exceeding 2.5%. Expressed as a percent of the lowest assay standard containing 2.9 mU FVIII/mL, the signals elicited by the 1/10-diluted serum samples were at least 23.3% lower. These data confirmed the selectivity of the antibody-based chromogenic FVIII assay for human FVIII as there was no evidence that endogenous animal proteins interfered with the assay.

### Example 7: Human FVIII Recovery in Citrated Laboratory Animal Plasma Samples

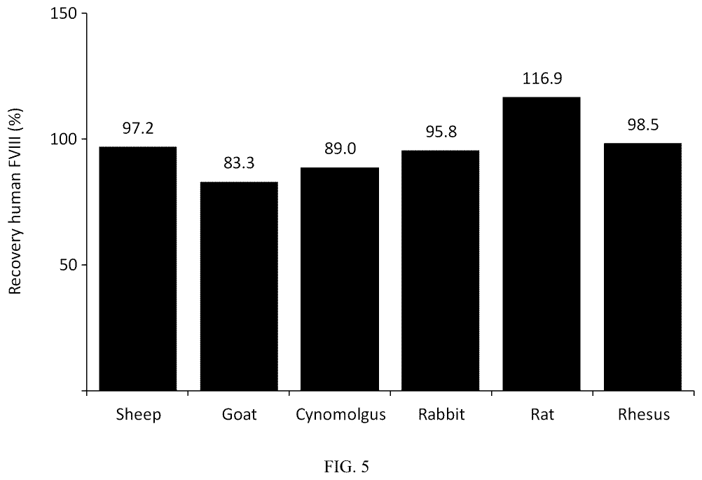

The citrated animal plasma samples were spiked with 100 mU human FVIII and measured in the final dilution of 1/10. The FVIII recoveries obtained were acceptable, i.e., within the commonly accepted 80-120% range [2]. and varied from 83.3% to 116.9% despite the low FVIII concentration of 100 mU/mL used for the spike-recovery experiment. These data confirmed the selectivity introduced by the antibody-driven capturing step as there was no signal from endogenous animal factor VIII, which would have clearly increased the recovery. Such elevated recoveries would have been observed when running a conventional chromogenic factor VIII assay that is not specific for human FVIII. FIG. 5 shows the individual recoveries determined for the human FVIII spiked to the citrated animal plasma species.

### Example 8: Human FVIII Recovery Analysis in Laboratory Animal Serum Samples

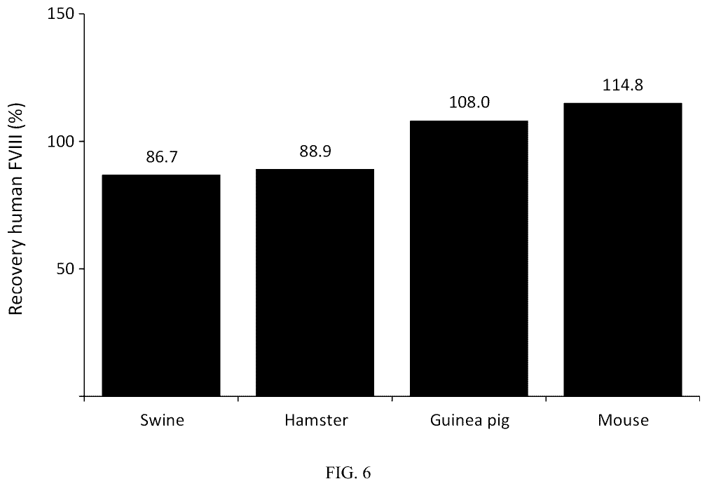

The animal serum samples were spiked with 100 mU human FVIII and measured in the final dilution of 1/10. FIG. 6 shows the recovery determined for the human FVIII spiked to the animal serum samples.

The recoveries were good, i.e., within the commonly accepted 80-120% range [2], and varied from 86.7% to 115.8% despite the low FVIII concentration of 100 mU/mL used for the spike-recovery experiment. The data confirmed the selectivity of the assay inasmuch there was no interference caused by the presence of animal serum proteins, introduced into the assay at relatively high concentrations for the major serum proteins including animal albumin and immunoglobulins.

### Example 9: Parallelism Investigation for Spiked Human FVIII in Animal Plasma and Serum Samples

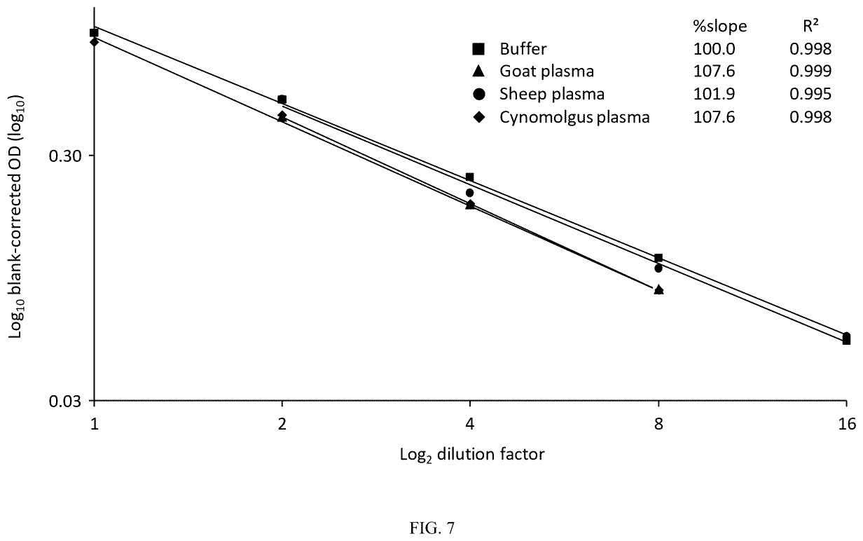

Adequate parallelism of the samples' concentration-response curves to that of the assay standard is a prerequisite, when the assay calibration curve is constructed with standards diluted in buffer. The use of buffer-based calibration curves, however, is intended to increase not only the assay's robustness, but also to lessen its complexity by reducing the number of biological reagents and by not requiring the construction of individual calibration curves for different animal matrices. Adequate parallelism between dilution series of the analyte in buffer and animal matrix demonstrating that there is no influence of the plasma/serum matrix can enable this simplification. Therefore, parallelism was checked using the results obtained for the plasma/serum samples spiked with 100 mU/mL human FVIII. FIG. 7 shows the dose-response curves for human FVIII spiked to citrated goat, sheep and cynomolgus monkey plasma.

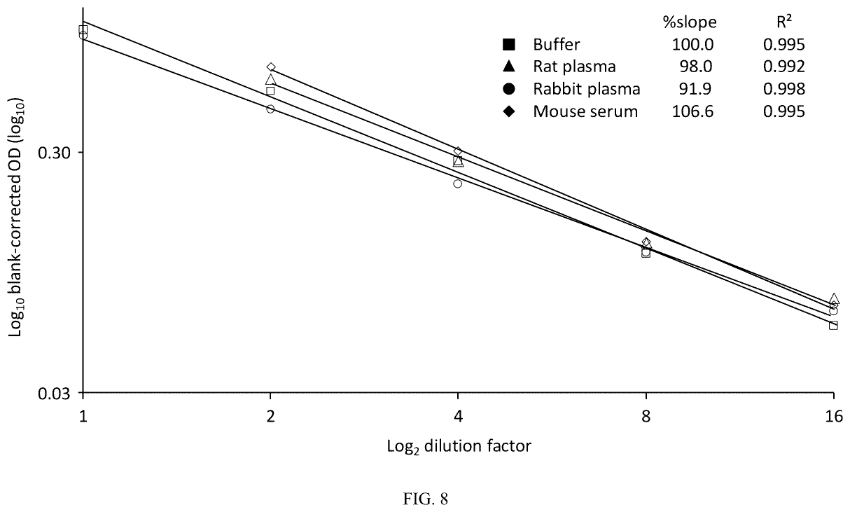

The slopes of the concentration-response curves for citrated goat, sheep and cynomolgus monkey plasma, starting at the dilution of 1/10, had slopes similar to that of the assay standard despite the presence of high concentrations of animal proteins. Thus, the slopes of the animal samples differed by less than 8% from that of the assay standard. Their linearity was almost identical, with all coefficients of determination being at least 0.995. These data demonstrated that there was most likely no essential influence of the animal protein matrix on the assay, thus supporting the important assay simplification of using a buffer-based calibration curve. FIG. 8 shows the dose-response curves for human FVIII spiked to citrated rabbit and rat plasma and mouse serum.

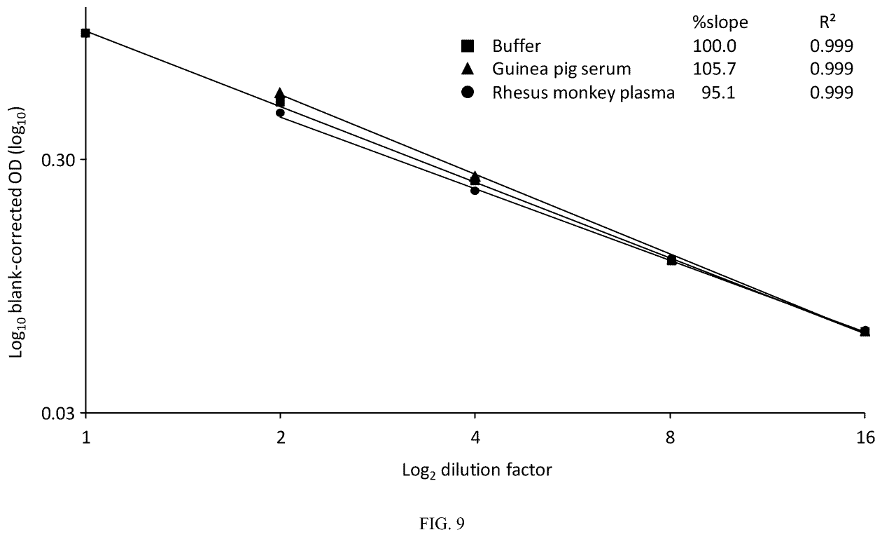

The slopes of the concentration-response curves for citrated rat and rabbit plasma and for mouse serum, starting at the dilution 1/10, had slopes similar to that of the assay standard with differences to the assay standard of less than 7%. Their linearity was almost identical, with coefficient of determinations being at least 0.992. These data demonstrated that there was most likely no essential influence of the animal protein matrix on the assay, thus supporting the important assay simplification of using a buffer-based calibration curve. FIG. 9) shows the dose-response curves for human FVIII spiked to citrated rhesus monkey plasma and guinea pig serum.

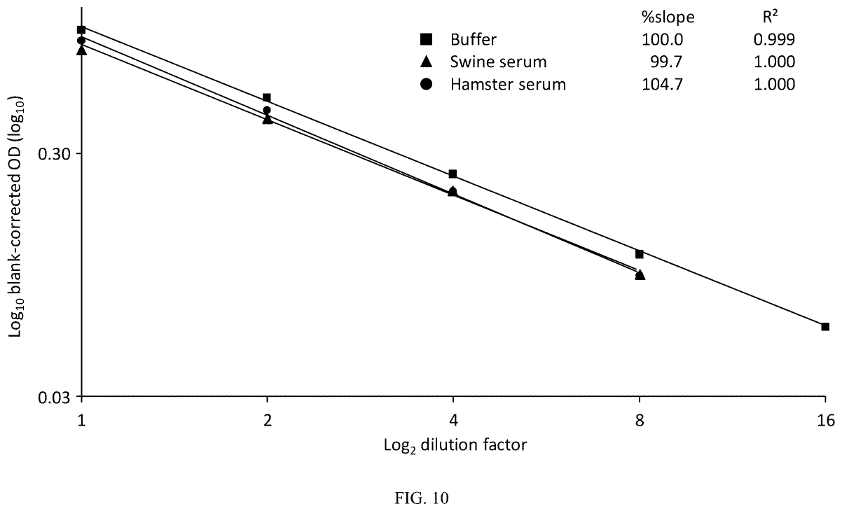

The slopes of the concentration-response curves for rhesus monkey plasma and guinea pig serum had slopes similar to that of the assay standard, with the slopes of the dose-response curves of the animal samples differing by less than 6% from that of the assay standard. Their linearity was identical (R2=0.999). These data demonstrated that there was most likely no essential influence of the animal protein matrix on the assay, thus supporting the important assay simplification of using a buffer-based calibration curve. FIG. 10 shows the dose-response curves for human FVIII spiked to swine and hamster serum.

The slopes of the concentration-response curves for swine and hamster serum had slopes similar to that of the assay standard, with the slopes of the animal samples differing by less than 5% from that of the assay standard. Their linearity was almost identical, with coefficient of correlations of at least 0.999. These data demonstrated that there was most likely no essential influence of the animal protein matrix on the assay, thus supporting the important assay simplification of using a buffer-based calibration curve.

### Example 10: Selected Assay Performance Characteristics in Cynomolgus Monkey Plasma

For this study, the assay was carried out as described in Example 1 using the reference plasma SSC/ISTH #04 with an assigned FVIII concentration of 0.88 IU/mL for the assay calibration instead of the Siemens reference plasma. This resulted in a slight increase in sensitivity with a calibration range from 2.8 to 88 mIU/mL, but had no impact on the quality attributes of the calibration curves. Among the performance characteristics in cynomolgus monkey plasma, selectivity for human FVIII measurement was confirmed by the measurement of citrated cynomolgus monkey plasma native and after having been spiked with human FVIII. This spike was carried out with recombinant human full length FVIII and human recombinant BDD FVIII. The spiked samples were used to calculate FVIII recovery as measure for assay accuracy, while their repeated analysis provided data on the assay's repeatability. Finally, the concentration-response curves obtained for the dilution series of the spiked samples allowed the evaluation of the parallelism between standard and samples. Table 6 shows the blank-corrected ODs, determined for six 1/10-dilutions of a citrated cynomolgus monkey plasma pool and of the corresponding assay standard D6, representing a FVIII concentration of 2.8 mIU/mL.

TABLE 6
Selectivity investigation for citrated cynomolgus monkey samples

| | | Blank-corrected optical densities | | | | |
|---|---|---|---|---|---|---|
| Plate | Dilution | [1] | [2] | [3] | Mean | D6 |
| 1 | 1/10 | −0.001 | 0.002 | 0.003 | 0.001 | 0.107 |
| 2 | 1/10 | −0.001 | −0.003 | −0.004 | −0.003 | 0.100 |

The assay standard D6 represented a FVIII concentration of 2.8 mIU/mL.

The six 1/10-diluted citrated cynomolgus monkey plasma samples had blank-corrected ODs close to zero with means of 0.001 and −0.003, while the corresponding assay standards containing the lowest FVIII concentration (2.8 mIU/mL), showed blank-corrected ODs of 0.107 and 0.100, respectively. Thus, the FVIII concentration found for citrated cynomolgus monkey plasma was <0.03 IU/mL, representing the assay's lower limit of quantification.

For the spike-recovery study in cynomolgus monkey plasma, seven parts of dilution buffer were mixed with two parts of citrated cynomolgus plasma and one part of diluted full length recombinant FVIII or recombinant BDD FVIII, representing FVIII concentrations of 1.63 and 1.76 IU/mL, respectively. These samples were then serially diluted 1+1 directly on the plate resulting in a final dilution of the cynomolgus monkey plasma of 1/10 and in nominal FVIII concentrations of 81.5 and 88.1 mIU/mL, respectively, for the samples spiked with full length and BDD IFVIII. Table 7 shows the recovery determined for full length and BDD IFVIII, spiked to citrated cynomolgus monkey plasma samples.

TABLE 7
Accuracy and precision study for
cynomolgus monkey plasma samples

| | Full-length rFVIII | | | BDD rFVIII | | |
|---|---|---|---|---|---|---|
| | Recovery | | RSD | Recovery | | RSD |
| Run | (%) | Mean | (%) | (%) | Mean | (%) |
| [1] | 100.1 | 102.7 | 2.8 | 89.9 | 92.4 | 2.4 |
| [2] | 102.2 | | | 93.0 | | |
| [3] | 105.9 | | | 94.2 | | |

All recoveries determined were within the generally accepted 100±20% range [2] with slightly better recoveries determined for the spiked full length rFVIII preparation. In particular, mean recoveries were 102.7% and 92.4% for the full length and BDD rFVIII preparation, respectively. The RSDs determined for the mean of the triplicate spikes did not exceed 2.8%. These data confirmed that accurate and precise measurement of human FVIII, without interference by endogenous monkey FVIII, can be achieved by the invented assay. Table 8 shows the slopes, the relative slopes (column “Slope %”) and the correlation coefficients of the dose-response curves.

TABLE 8
Parallelism study for cynomolgus monkey plasma samples

| | Full length rFVIII | | | | BDD rFVIII | | | |
|---|---|---|---|---|---|---|---|---|
| Attribute | Standard | [1] | [2] | [3] | Standard | [1] | [2] | [3] |
| Slope | −0.8447 | −0.8701 | −0.8922 | −0.8952 | −0.8542 | −0.8559 | −0.8658 | −0.8568 |
| R² | 0.998 | 0.999 | 0.999 | 0.999 | 0.997 | 0.999 | 0.997 | 0.999 |
| Slope % | 100.0 | 103.0 | 105.6 | 106.0 | 100.0 | 100.2 | 101.4 | 100.3 |

The row “Slope %” shows the relative slopes, which are expressed as a percent of the slope of the dose-response curve of the corresponding assay standard.

The slopes determined for the dose-response curves of the spiked monkey plasma samples differed by not more than 6% from that of the assay standard. The mean relative slopes of the dose-response curves were 104.9% and 100.5% for the plasma samples spiked with full length and BDD rFVIII, respectively.

### Example 11: Selected Assay Performance Characteristics in Buffer

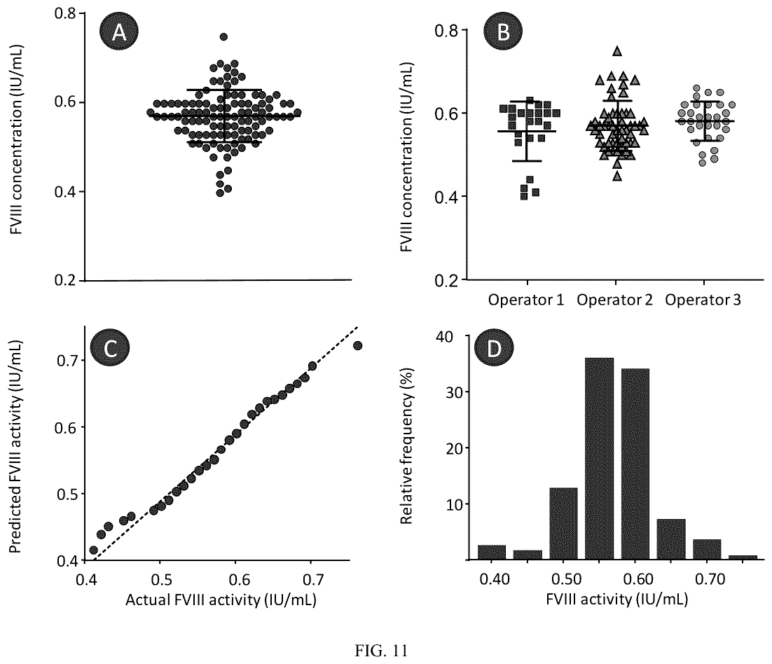

The lyophilized human reference plasma preparation CRYOcheck (Precision BioLogic) was used as assay control. FIG. 11A-11D shows the data of 108 measurements, carried out by three operators over a time period of six months to demonstrate precision and accuracy over this time period.

The mean obtained by 108 measurements carried out by three operators over a six-month period was 0.57±0.06 IU/mL (RSD 10.4%). The data were normal distributed as checked with D'Agostino & Pearson test (p=0.1174; GraphPad Prism 8.3). ANOVA calculated with GraphPad Prism 8.3 showed that there was no statistically significant difference between the results obtained by the three operators (p=0.3407). These data confirmed adequate intermediate precision and robustness of the assay over an extended time of six months. Table 9 shows the results of a repeatability study, done with a full length and BDD FVIII preparation.

TABLE 9
Repeatability analysis for rFVIII preparations

| | FVIII activity in IU/mL | | | | | | | RSD |
|---|---|---|---|---|---|---|---|---|
| Sample | [1] | [2] | [3] | [4] | [5] | [6] | Mean | (%) |
| Full length | 33.4 | 32.9 | 31.9 | 30.9 | 33.1 | 33.3 | 32.6 | 3.0 |
| rFVIII | | | | | | | | |
| BDD rFVIII | 1805 | 1769 | 1780 | 1710 | 1753 | 1747 | 1761 | 1.8 |

Both rFVIII preparations had FVIII activities of higher than 30 IU/mL and thus, the serial dilution series prepared for the measurement of the full length and BDD IFVIII preparation started at dilutions 1/400 and 1/20000, respectively. The RSDs obtained for the six-fold measurements in one run were low: RSDs of 3.0% and 1.8% were determined for the full length and the BDD rFVIII, respectively. These results thus met general precision requirements for bioanalytical methods [2].

### Example 12: Influence of Solvent-Detergent Virus Inactivation Solution on the Antibody-Based Chromogenic FVIII Assay

The antibody-based capturing of FVIII followed by the chromogenic activity measurement provides an additional option to measure FVIII activity in sample matrices so far not suitable for the measurement because of considerable interference with the assay. An advantage of the invented assay is that substances interfering with the chromogenic assay can be removed by washing the captured FVIII, as long as there is no effect on the capturing step.

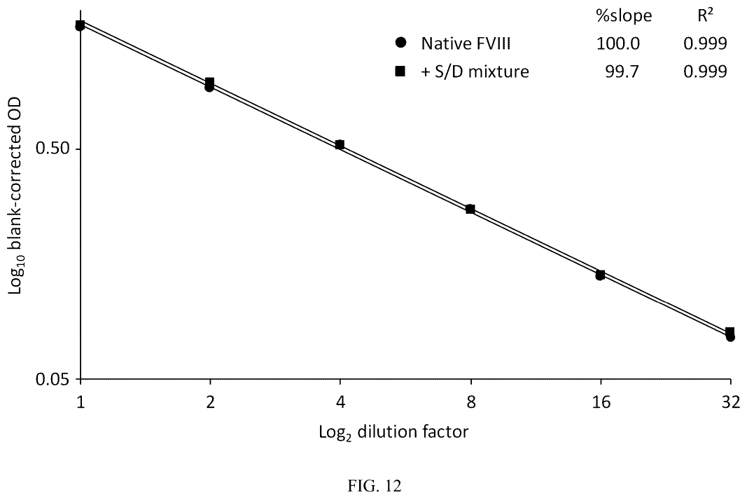

The solvent detergent (S/D) solution, often used as an effective virus inactivation solution and containing 1% Triton X-100, 0.3% Polysorbate 80 and 0.3% tri-n-butyl phosphate (tnBP), is an example of a specific matrix being incompatible with the chromogenic activity assay. The presence of the components of the S/D solution, however, was not expected to interfere with the capture of FVIII by the plate-bound antibody. FIG. 12 shows the concentration-response curves obtained for a sample containing 10 IU human FVIII/mL in the presence of the S/D solution, i.e., 1% Triton X 100, 0.3% Polysorbate 80 and 0.3% tnBP, and the same FVIII concentration in PBS buffer (=native FVIII).

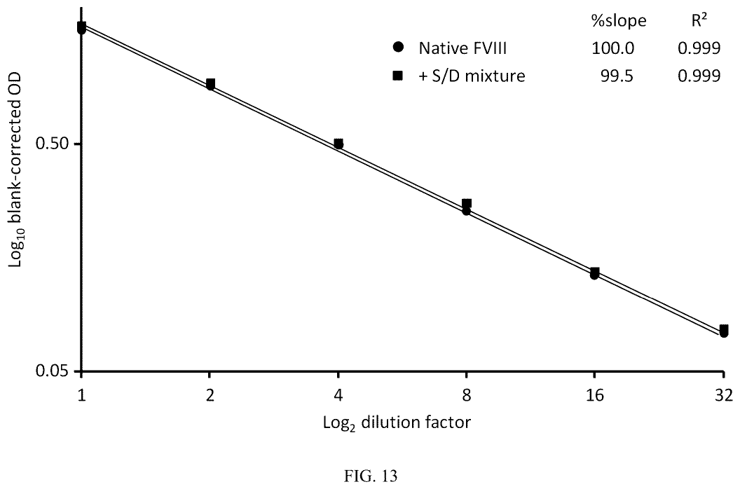

Almost identical dilution-response curves obtained for FVIII in S/D solution and buffer demonstrated that the S/D mixture had no influence on the assay performance. Slopes and squared correlation coefficients demonstrated the similarity between both samples. FIG. 13 shows the results of a similar experiment but using a FVIII concentration of 1 IU/mL only, requiring a 10-times lower sample dilution and thus markedly increasing the levels of S/D components during the antibody-based FVIII capture.

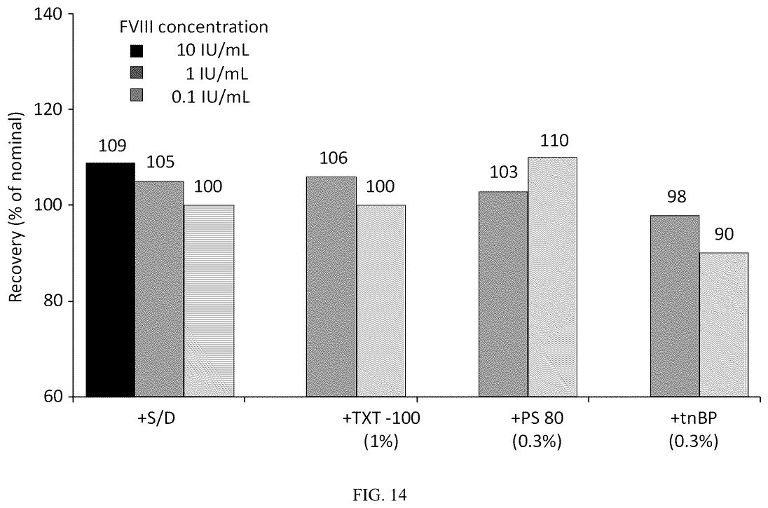

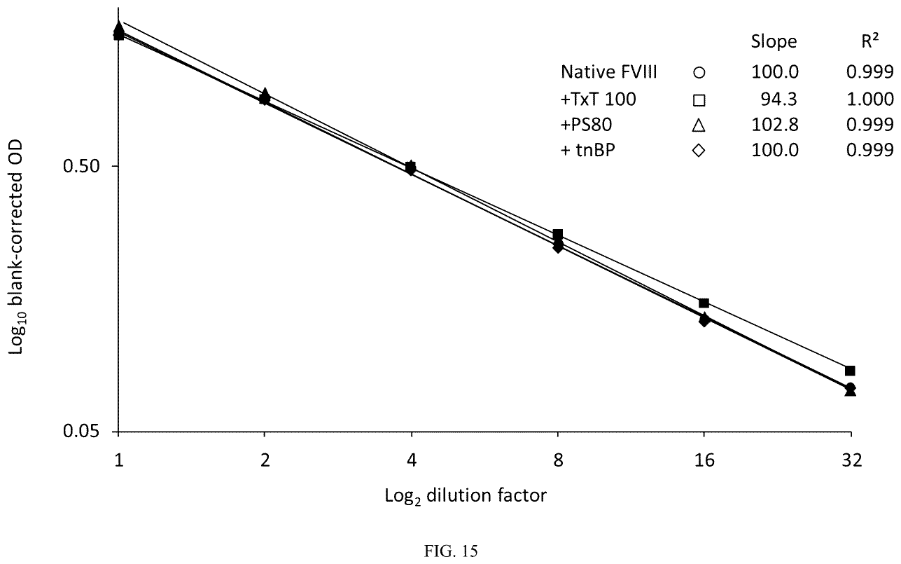

Under the conditions using the lower concentration of FVIII, much higher concentrations of the S/D components were present during the initial capture step, and both dilution-response curves were highly similar in slope and linearity. Thus, the presence of S/D solution had minimal to no impact on the assay performance. FIG. 14 shows the recovery of three levels of human FVIII (10, 1 and 0.1 IU/mL) in S/D solution and the individual components of the S/D solution. FIG. 15 shows assay performance in presence of individual components of the S/D virus inactivation solution.

### References

[1] Rotblat F, Goodall AH, O'Brien DP, Rawlings E, Middleton S, Tuddenham EGD (1983): Monoclonal antibodies to human procoagulant factor VIII. J Lab Clin Med 101, 736-46.
[2] EMEA/CHMP/EWP/192217/2009. Bioanalytical assay validation (2011).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for measuring the activity of human coagulation factor VIII (FVIII) in a sample, wherein the sample comprises one or more FVIII-activity mimicking agents, said method comprising the steps of:
a) incubating the sample with a capture agent that selectively binds the human FVIII under conditions and for a time sufficient to allow the capture agent to selectively bind to the human FVIII in the presence of the one or more FVIII-activity mimicking agents, whereby forming a reaction mixture, wherein the capture agent is attached to a solid support;
b) removing unbound fraction of human FVIII from the reaction mixture; and
c) measuring the activity of human FVIII in the reaction mixture using a chromogenic assay.

2. The method of claim 1, further comprising d) determining the activity of human FVIII in the reaction mixture by comparing with an FVIII reference preparation.

3. The method of claim 1, wherein the chromogenic assay is performed by
c1) adding to the reaction mixture activated factor IX (FIXa), factor X (FX), calcium ion, phospholipids, and thrombin, and incubating the reaction mixture under conditions and for a time sufficient to generate activated FX (FXa);
c2) adding to the reaction mixture an FXa specific chromogenic substrate and incubating the reaction mixture under conditions and for a time sufficient to generate a measurable signal;
c3) optionally, adding to the reaction mixture a stopping agent to stop signal generation from the FXa specific chromogenic substrate; and
c4) measuring the signal generated from the reaction mixture.

4. The method of claim 3, wherein the chromogenic substrate is CH₃OCO-D-cyclohexylalanine-Gly-Arg-pNA, and/or the stopping agent is acetic acid.

5. (canceled)

6. The method of claim 1, wherein the capture agent selectively binds to the A1, A2, A3, B, C1, or C2 domain of human FVIII.

7. The method of claim 1, wherein the capture agent selectively binds to the A2 domain of human FVIII.

8. The method of claim 1, wherein the capture agent does not bind the B domain of human FVIII.

9. The method of claim 1, wherein the capture agent is an antibody, an antigen-binding fragment, an affibody, an aptamer or an affinity ligand.

10. The method of claim 9, wherein the capture antibody is GMA-8024, GMA-8023, or an antigen-binding fragment or a variant thereof.

11. The method of claim 1, wherein the sample comprises plasma and/or serum from a non-human animal or a laboratory animal.

12. (canceled)

13. The method of claim 11, wherein the non-human animal is sheep, goat, cynomolgus monkey, rabbit, rat, rhesus macaque, mouse, swine, hamster, minipig, or guinea pig.

14. (canceled)

15. (canceled)

16. The method of claim 1, wherein the FVIII-activity mimicking agent is an antibody, an antigen-binding fragment, a peptide, a nucleic acid, or a small molecule.

17. The method of claim 16, wherein the FVIII-activity mimicking agent is a bispecific antibody.

18. The method of claim 17, wherein the bispecific antibody is Emicizumab.

19. The method of claim 1, wherein
(1) the sample is diluted prior to step a);
(2) the incubation in step a) is performed for about 1 hour at room temperature;
(3) the incubation in step c1) is performed for about 15 min at room temperature;
(4) the incubation in step c2) is performed for about 25 min at room temperature; and/or
(5) the removal of unbound fraction of human FVIII in step b) is performed by washing the solid support with a washing buffer.

20. The method of claim 19, wherein the sample is diluted by a factor of between 1:1 to about 1:100,000, between 1:2 to about 1:20,000, or about 1:10.

21. (canceled)

22. (canceled)

23. The method of claim 1, wherein the solid support is a flask, ELISA plate, multi-well plate, a film, a tube, a sheet, a column, or a microparticle.

24-28. (canceled)

29. The method of claim 1, wherein the human FVIII is a recombinant FVIII.

30. The method of claim 1, wherein the human FVIII is full-length FVIII, or B-domain deleted FVIII.

31. (canceled)

32. A kit for measuring the activity of human coagulation factor VIII (FVIII) in a sample, comprising
i. a capture agent that selectively binds the human FVIII, wherein the capture agent is attached to a solid support;
ii. a first vial comprising one or more of activated factor IX (FIXa), factor X (FX), calcium ion, phospholipids and thrombin;
iii. a second vial comprising an FXa specific chromogenic substrate; and/or
iv. optionally, a third vial comprising a stopping agent.

33-41. (canceled)
